SEQUENCE LISTING (105 sequences)

SEQ ID NO: 1 - protein, 20 aa, Artificial Sequence
  Synthetic polypeptide
RRGMKQYERISRDANRSYRR

SEQ ID NO: 2 - protein, 20 aa, Artificial Sequence
  Synthetic polypeptide
RQYMRQIEQALRYGYRISRR

SEQ ID NO: 3 - protein, 20 aa, Artificial Sequence
  Synthetic polypeptide
RKYMRQYEEAIRDGNRSIRR

SEQ ID NO: 4 - protein, 20 aa, Artificial Sequence
  Synthetic polypeptide
RQYMRYLEQAERYVNRNLRR

SEQ ID NO: 5 - protein, 20 aa, Artificial Sequence
  Synthetic polypeptide
RKLMEMYEEAFRYFNRISRR

SEQ ID NO: 6 - protein, 20 aa, Artificial Sequence
  Synthetic polypeptide
RSIMELYKQASRSFNRGIRR

SEQ ID NO: 7 - protein, 20 aa, Artificial Sequence
  Synthetic polypeptide
RQIYESIEQALRRGYRSYRR

SEQ ID NO: 8 - protein, 20 aa, Artificial Sequence
  Synthetic polypeptide
RSYYEAYERALRKGQRGIRR

SEQ ID NO: 9 - protein, 20 aa, Artificial Sequence
  Synthetic polypeptide
RAYMEALRQAERLGNRTARR

SEQ ID NO: 10 - protein, 20 aa, Artificial Sequence
  Synthetic polypeptide
RYLMEYAEQAKRDAKRAYRR

SEQ ID NO: 11 - protein, 20 aa, Artificial Sequence
  Synthetic polypeptide
RQLMELIEQAERYGNRFYRR

SEQ ID NO: 12 - protein, 20 aa, Artificial Sequence
  Synthetic polypeptide
RKLMELYEQAIRYGKRSYRR

SEQ ID NO: 13 - protein, 20 aa, Artificial Sequence
  Synthetic polypeptide
RRYMECYEQAERYFRRFGRR

SEQ ID NO: 14 - protein, 20 aa, Artificial Sequence
  Synthetic polypeptide
RSFMKCYEQASRYGNRILRR

SEQ ID NO: 15 - protein, 20 aa, Artificial Sequence
  Synthetic polypeptide
RKLVECYERAERDANRSGRR

SEQ ID NO: 16 - protein, 20 aa, Artificial Sequence
  Synthetic polypeptide
RQLMECYEQAARRGARSYRR

SEQ ID NO: 17 - protein, 20 aa, Artificial Sequence
  Synthetic polypeptide
RYMMKIYEQAERYFNRVGRR

SEQ ID NO: 18 - protein, 20 aa, Artificial Sequence
  Synthetic polypeptide
RRYYEQLEQASRKGNRGFRR

SEQ ID NO: 19 - protein, 20 aa, Artificial Sequence
  Synthetic polypeptide
RSVMEQYEQAARDAYRSARR

SEQ ID NO: 20 - protein, 20 aa, Artificial Sequence
  Synthetic polypeptide
RQYMECIEKALRDGYRSYRR

SEQ ID NO: 21 - protein, 20 aa, Artificial Sequence
  Synthetic polypeptide
RYYMKCYKQAARYIYRGYRR

SEQ ID NO: 22 - protein, 20 aa, Artificial Sequence
  Synthetic polypeptide
RSAYEYYRRAYRDGNRGYRR

SEQ ID NO: 23 - protein, 20 aa, Artificial Sequence
  Synthetic polypeptide
RYGMRQFEQASRDGNRSFRR

SEQ ID NO: 24 - protein, 20 aa, Artificial Sequence
  Synthetic polypeptide
RKGYRGYEQALRYGKRYGRR

SEQ ID NO: 25 - protein, 20 aa, Artificial Sequence
  Synthetic polypeptide
RYGMRCLEEALRYGNRGYRR

SEQ ID NO: 26 - protein, 20 aa, Artificial Sequence
  Synthetic polypeptide
RQYREIIEQARRVGNRGARR

SEQ ID NO: 27 - protein, 20 aa, Artificial Sequence
  Synthetic polypeptide
RQGMEVYERASRQGNRSLRR

SEQ ID NO: 28 - protein, 20 aa, Artificial Sequence
  Synthetic polypeptide
RRIMEQYEEAERDGNRVYRR

SEQ ID NO: 29 - protein, 20 aa, Artificial Sequence
  Synthetic polypeptide
RQVMEAYEQFYRDGNRAYRR

SEQ ID NO: 30 - protein, 20 aa, Artificial Sequence
  Synthetic polypeptide
RQLMEQYEQAYRYAARGYRR

SEQ ID NO: 31 - protein, 20 aa, Artificial Sequence
  Synthetic polypeptide
RYIMEIYEQAIRKGNRSYRR

SEQ ID NO: 32 - protein, 20 aa, Artificial Sequence
  Synthetic polypeptide
RKYMELYEKASRRGYRGYRR

SEQ ID NO: 33 - protein, 20 aa, Artificial Sequence
  Synthetic polypeptide
RQYLEQYENAERYIYRAYRR

SEQ ID NO: 34 - protein, 20 aa, Artificial Sequence
  Synthetic polypeptide
RQYMKCYEQAYRYGRRGYRR

SEQ ID NO: 35 - protein, 20 aa, Artificial Sequence
  Synthetic polypeptide
RQYAEQYEEAIRDGNRSVRR

SEQ ID NO: 36 - protein, 20 aa, Artificial Sequence
  Synthetic polypeptide
RSYMEMLEQIERYGNRVGRR

SEQ ID NO: 37 - protein, 20 aa, Artificial Sequence
  Synthetic polypeptide
RQYMEFVEQAERYGRRGSRR

SEQ ID NO: 38 - protein, 20 aa, Artificial Sequence
  Synthetic polypeptide
RSYMEQYEEAIRRGYRSYRR

SEQ ID NO: 39 - protein, 20 aa, Artificial Sequence
  Synthetic polypeptide
RQYMKYYEEAERYGNRAYRR

SEQ ID NO: 40 - protein, 20 aa, Artificial Sequence
  Synthetic polypeptide
RAYMEYYEQFYRMGKRASRR

SEQ ID NO: 41 - protein, 20 aa, Artificial Sequence
  Synthetic polypeptide
RQYMEQVEQALRDGYRSGRR

SEQ ID NO: 42 - protein, 20 aa, Artificial Sequence
  Synthetic polypeptide
RSYMESIEQALRIGNRSYRR

SEQ ID NO: 43 - protein, 20 aa, Artificial Sequence
  Synthetic polypeptide
RSYMEIYEQASRAGNRAYRR

SEQ ID NO: 44 - protein, 20 aa, Artificial Sequence
  Synthetic polypeptide
RQYMEYYEQVFRAGYRSARR

SEQ ID NO: 45 - protein, 20 aa, Artificial Sequence
  Synthetic polypeptide
RYYMECYEQAVRYGRRWYRR

SEQ ID NO: 46 - protein, 20 aa, Artificial Sequence
  Synthetic polypeptide
RQGMECYEQALRYGQRGIRR

SEQ ID NO: 47 - protein, 20 aa, Artificial Sequence
  Synthetic polypeptide
RSFMEQGEQAFRDGYRMYRR

SEQ ID NO: 48 - protein, 20 aa, Artificial Sequence
  Synthetic polypeptide
RKYMEIYEKASRYGNRSYRR

SEQ ID NO: 49 - protein, 20 aa, Artificial Sequence
  Synthetic polypeptide
RQYKEAYEEIYRYGNRMGRR

SEQ ID NO: 50 - protein, 20 aa, Artificial Sequence
  Synthetic polypeptide
RRYMECYEQAERDGNRMYRR

SEQ ID NO: 51 - protein, 20 aa, Artificial Sequence
  Synthetic polypeptide
RAYMECLEQAERYGNRAYRR

SEQ ID NO: 52 - protein, 20 aa, Artificial Sequence
  Synthetic polypeptide
RQVMETYEQLERYGNRSARR

SEQ ID NO: 53 - protein, 20 aa, Artificial Sequence
  Synthetic polypeptide
RQIRECYEQASRYGNRSYRR

SEQ ID NO: 54 - protein, 20 aa, Artificial Sequence
  Synthetic polypeptide
RQYMEVYEQAERAGNRVYRR

SEQ ID NO: 55 - protein, 20 aa, Artificial Sequence
  Synthetic polypeptide
RSYMEQYEQAFRRGNRSYRR

SEQ ID NO: 56 - protein, 20 aa, Artificial Sequence
  Synthetic polypeptide
RHFMECYEQASRDGNRSLRR

SEQ ID NO: 57 - protein, 20 aa, Artificial Sequence
  Synthetic polypeptide
RKAMEQYEEAERDGARSYRR

SEQ ID NO: 58 - protein, 20 aa, Artificial Sequence
  Synthetic polypeptide
RQYMKGYEQAERHAYRSYRR

SEQ ID NO: 59 - protein, 20 aa, Artificial Sequence
  Synthetic polypeptide
RQYMEQAEQAERDGNRSVRR

SEQ ID NO: 60 - protein, 20 aa, Artificial Sequence
  Synthetic polypeptide
RSIMEYYEQIERDGNRSYRR

SEQ ID NO: 61 - protein, 20 aa, Artificial Sequence
  Synthetic polypeptide
RYLKECYEQASRIGYRGLRR

SEQ ID NO: 62 - protein, 20 aa, Artificial Sequence
  Synthetic polypeptide
RQGMEAYEQAERLGNRGIRR

SEQ ID NO: 63 - protein, 20 aa, Artificial Sequence
  Synthetic polypeptide
RQYMECYKQIYRYGNRSYRR

SEQ ID NO: 64 - protein, 20 aa, Artificial Sequence
  Synthetic polypeptide
RSYREYAEQALRYGNRGYRR

SEQ ID NO: 65 - protein, 20 aa, Artificial Sequence
  Synthetic polypeptide
RSGMEYYKQAFRAGYRVTRR

SEQ ID NO: 66 - protein, 20 aa, Artificial Sequence
  Synthetic polypeptide
RSAMECYEKAERYWYRGSRR

SEQ ID NO: 67 - protein, 20 aa, Artificial Sequence
  Synthetic polypeptide
RSYMECYEQASRKGNRSIRR

SEQ ID NO: 68 - protein, 20 aa, Artificial Sequence
  Synthetic polypeptide
RQYMELYEQAMRYGNRGYRR

SEQ ID NO: 69 - protein, 20 aa, Artificial Sequence
  Synthetic polypeptide
RQYIECYEQAARYGKRGYRR

SEQ ID NO: 70 - protein, 20 aa, Artificial Sequence
  Synthetic polypeptide
RQWAEYYEQLERYGNRSYRR

SEQ ID NO: 71 - protein, 20 aa, Artificial Sequence
  Synthetic polypeptide
RSYMEAYEQASRDGYRLYRR

SEQ ID NO: 72 - protein, 20 aa, Artificial Sequence
  Synthetic polypeptide
RQYMEQYEQFERAGNRVYRR

SEQ ID NO: 73 - protein, 20 aa, Artificial Sequence
  Synthetic polypeptide
RYYMEYYEKASRYGNRGIRR

SEQ ID NO: 74 - protein, 20 aa, Artificial Sequence
  Synthetic polypeptide
RYYMEYYEQLERYGNRLYRR

SEQ ID NO: 75 - protein, 20 aa, Artificial Sequence
  Synthetic polypeptide
RQYMECYEQAARYGNRSYRR

SEQ ID NO: 76 - protein, 20 aa, Artificial Sequence
  Synthetic polypeptide
RQYMEIYEQASRYGNRSYRR

SEQ ID NO: 77 - protein, 20 aa, Artificial Sequence
  Synthetic polypeptide
RQYMEQYEQAMRDGNRGYRR

SEQ ID NO: 78 - protein, 20 aa, Artificial Sequence
  Synthetic polypeptide
RQYMEYYEQFSRLGNRSYRR

SEQ ID NO: 79 - protein, 20 aa, Artificial Sequence
  Synthetic polypeptide
RSGMKVYEQAERYGNRSYRR

SEQ ID NO: 80 - protein, 20 aa, Artificial Sequence
  Synthetic polypeptide
RSAMECYEKASRDGNRGSRR

SEQ ID NO: 81 - protein, 20 aa, Artificial Sequence
  Synthetic polypeptide
RYYKEYYEKAERIGNRGYRR

SEQ ID NO: 82 - protein, 20 aa, Artificial Sequence
  Synthetic polypeptide
RSYMECYEQAFRYGKRSSRR

SEQ ID NO: 83 - protein, 20 aa, Artificial Sequence
  Synthetic polypeptide
RQYMECYKQAERYGNRGYRR

SEQ ID NO: 84 - protein, 20 aa, Artificial Sequence
  Synthetic polypeptide
RSVMEYYEQAYRYGNRGSRR

SEQ ID NO: 85 - protein, 20 aa, Artificial Sequence
  Synthetic polypeptide
RQGMEAYEQAERYGNRSYRR

SEQ ID NO: 86 - protein, 20 aa, Artificial Sequence
  Synthetic polypeptide
RAYQEAYEQAYRDGNRSYRR

SEQ ID NO: 87 - protein, 20 aa, Artificial Sequence
  Synthetic polypeptide
RSYMEQYEQASRKGYRSYRR

SEQ ID NO: 88 - protein, 20 aa, Artificial Sequence
  Synthetic polypeptide
RSYAECYEQISRYGNRGYRR

SEQ ID NO: 89 - protein, 20 aa, Artificial Sequence
  Synthetic polypeptide
RSYMEAYEQAERYGNRGYRR

SEQ ID NO: 90 - protein, 20 aa, Artificial Sequence
  Synthetic polypeptide
SQRVEQYVRRLYDDYRNYMR

SEQ ID NO: 91 - protein, 20 aa, Artificial Sequence
  Synthetic polypeptide
RSYIEQYEQLERDGARSYRR

SEQ ID NO: 92 - protein, 20 aa, Artificial Sequence
  Synthetic polypeptide
SQRLERYVERSFDDYRKSGR

SEQ ID NO: 93 - protein, 20 aa, Artificial Sequence
  Synthetic polypeptide
RSYMEYYEQASRDGARGYRR

SEQ ID NO: 94 - protein, 20 aa, Artificial Sequence
  Synthetic polypeptide
SKRVGQGVERSYKKYRNYIR

SEQ ID NO: 95 - protein, 20 aa, Artificial Sequence
  Synthetic polypeptide
GQRVEQLVERYGDDLRNSVR

SEQ ID NO: 96 - protein, 20 aa, Artificial Sequence
  Synthetic polypeptide
YQRVEQYVQRSYDAYRNYAR

SEQ ID NO: 97 - protein, 20 aa, Artificial Sequence
  Synthetic polypeptide
SQRVEQYVERYADGYRNYLR

SEQ ID NO: 98 - protein, 20 aa, Artificial Sequence
  Synthetic polypeptide
YQRVEQYVQRYHDDLRNYSR

SEQ ID NO: 99 - protein, 20 aa, Artificial Sequence
  Synthetic polypeptide
YQRVEQYVQRSYDDYRNVGR

SEQ ID NO: 100 - protein, 20 aa, Artificial Sequence
  Synthetic polypeptide
TQRVEQYVERSSDKYRNLGR

SEQ ID NO: 101 - protein, 20 aa, Artificial Sequence
  Synthetic polypeptide
SSRMECYEQAERYGYGGYGG

SEQ ID NO: 102 - protein, 20 aa, Artificial Sequence
  Synthetic polypeptide
RYGYGGYGGGRYGGGYGSGR

SEQ ID NO: 103 - protein, 20 aa, Artificial Sequence
  Synthetic polypeptide
YGYGGYGGGRYGGGYGSGRG

SEQ ID NO: 104 - protein, 20 aa, Artificial Sequence
  Synthetic polypeptide
GQPVGQGVERSHDDNRNQPR

SEQ ID NO: 105 - protein, 23 aa, Artificial Sequence
  Synthetic polypeptide
GIGKFLHSAKKFGKAFVGEIMNS

(19) United States
(12) Patent Application Publication
(10) Pub. No.: US 2021/0183469 A9
(43) Pub. Date: Jun. 17, 2021

(54) COMPUTATIONAL PLATFORM FOR IN SILICO COMBINATORIAL SEQUENCE SPACE EXPLORATION AND ARTIFICIAL EVOLUTION OF PEPTIDES

(71) Applicants: Massachusetts Institute of Technology, Cambridge, MA (US); Universidade Católica de Brasília, Aguas Claras (BR)

(72) Inventors: Timothy Kuan-Ta Lu, Cambridge, MA (US); Cesar De la Fuente Nunez, Somerville, MA (US); William Porto, Brasília-DF (BR); Octavio Franco, Brasília-DF (BR)

(73) Assignees: Massachusetts Institute of Technology, Cambridge, MA (US); Universidade Católica de Brasília, Aguas Claras (BR)

(21) Appl. No.: 16/299,641

(22) Filed: Mar. 12, 2019

Related U.S. Application Data
(60) Provisional application No. 62/641,513, filed on Mar. 12, 2018.

(51) Int. Cl. G16B 35/10; C07K 14/00; A61P 31/04

(52) U.S. Cl. CPC G16B 35/10; A61K 38/00; A61P 31/04; C07K 14/001

(57) ABSTRACT

Disclosed herein are methods of designing peptides having at least one property of interest, such as α-helical propensity, higher net charge, hydrophobicity, and/or hydrophobic moment. Also disclosed herein are novel artificially evolved peptides (e.g., antimicrobial peptides), which may be designed according to the methods described herein, and methods of use thereof.

### RELATED APPLICATION

This application claims the benefit under 35 U.S.C. § 119(e) of U.S. Provisional Application No. 62/641,513, filed on Mar. 12, 2018, and entitled “Computational Platform for In Silico Combinatorial Sequence Space Exploration and Artificial Evolution of Peptides,” which is incorporated herein by reference in its entirety for all purposes.

### GOVERNMENT SUPPORT

This invention was made with Government support under Grant No. HDTRA1-15-1-0050 awarded by the Defense Threat Reduction Agency (DTRA). The Government has certain rights in the invention.

### FIELD

Disclosed herein are methods of designing peptides having at least one property of interest, such as α-helical propensity, higher net charge, hydrophobicity, and/or hydrophobic moment. Also disclosed herein are novel artificially evolved peptides (e.g., antimicrobial peptides), which may be designed according to the methods described herein, and methods of use thereof.

### BACKGROUND

Hospital-acquired infections are a major global health concern and represent the sixth leading cause of death in the United States, with an estimated cost of ˜$10 billion annually (Peleg & Hooper, N. Engl. J. Med. 2010 May 13; 362(19): 1804-13). Infections caused by Gram-negative bacteria such as Pseudomonas aeruginosa have been associated with more than 60% of pneumonia cases and more than 70% of urinary tract infections in intensive care units (Gaynes & Edwards, Clin. Infect. Dis. 2005 Sep. 15; 41(6): 848-54). Additionally, such bacteria are highly efficient in generating mutants and sharing genes that encode antibiotic resistance (Peleg & Hooper, N. Engl. J. Med. 2010 May 13; 362(19): 1804-13). It has been recently estimated that 30 million sepsis cases occur worldwide each year as a result of antibiotic-resistant infections, potentially leading to 5 million deaths (Fleischmann et al., Am. J. Respir. Crit. Care Med. 2016 Feb. 1; 193(3): 259-72.). Therefore, there is an urgent need to develop alternatives to antibiotics, particularly against Gram-negative bacteria, and advance new strategies to combat bacterial resistance. Unfortunately, in the past two decades only two novel classes of antibiotics have reached the market, oxazolidinones and cyclic lipopeptides, and both of these drugs are limited as they only target Gram-positive bacteria (Coates et al., Br. J. Pharmacol. 2011 May; 163(1): 184-94).

### SUMMARY

AMPs have been proposed as a promising alternative to conventional antibiotics and are considered potential next-generation antimicrobial agents (Brogden, Nat. Rev. Microbiol. 2005 March; 3(3): 238-50; Fjell et al., Nat. Rev. Drug Discov. 2011 Dec. 16; 11(1): 37-51). The development of AMPs into drugs, however, has been limited by their high design cost and the inability to rationally manipulate these agents. In addition, although known AMPs show redundancy in their primary sequence, their potential natural sequence space (20n, n being the number of residues in a peptide chain) suggests an almost unlimited number of amino acid combinations that may be exploited to generate completely novel synthetic peptides different from any that exist in nature. Novel computational approaches may enable exploration of the combinatorial sequence space of AMPs thus reducing the design cost of these agents, and may yield completely novel molecules with unprecedented antimicrobial activity.

Antimicrobial peptides (AMPs) represent promising alternatives to conventional antibiotics, yet the translation of AMPs into the clinic is hindered by high costs of design and synthesis. Described herein is a computational platform for streamlining AMP design, based on a genetic algorithm that exploits a sequence space different from that of previously described AMPs. This approach, as demonstrated herein, is effective for designing peptide antibiotics. Implementing this approach yielded guavanins, synthetic peptides having an unusually high proportion of arginines, and tyrosines as hydrophobic counterparts, which are also disclosed herein.

Accordingly, in some aspects, the disclosure relates to methods of designing peptides having at least one property of interest. In some embodiments, the method comprises: (a) selecting a population of parent peptides; (b) calculating a fitness function value for each peptide in the population of peptides of (a), wherein the fitness function value is indicative of the presence of at least one property of interest; (c) selecting a fraction of the peptides from the population of peptides, wherein the fitness function values of the selected fraction of peptides are higher than the fitness function values of the non-selected fraction of peptides; (d) subjecting the fraction of peptides in (c) to fitness-guided mutation comprising at least a single point cross over and at least a 0.05% probability of mutation, thereby generating a population of mutated peptides; (e) calculating a fitness function value for each peptide in the population of mutated peptides of (d), wherein the fitness function value is indicative of the presence of the at least one property of interest in (b); and (f) iteratively repeating steps (c)-(e), wherein the number of iterations does not result in the plateauing of the average fitness function values of the population of selected peptides of (e).

In some embodiments, the peptides in the population of parent peptides in (a) consist of the same amino acid sequence. In some embodiments, the peptides in the population of parent peptides in (a) comprise two or more amino acid sequences.

In some embodiments, each peptide in the population of parent peptides in (a) has essentially the same fitness function value. In some embodiments, the fitness function is represented by the equation:

$$Fitness = \frac{\sqrt[2]{{[ {\sum }_{i = 1}^{I} {H}_{i} \times cos ( δ i ) ]}^{2} + {[ {\sum }_{i = 1}^{I} {H}_{i} \times sin ( δ i ) ]}^{2}}}{{\sum }_{i = 1}^{I} {e}^{{Hx}_{i}}}$$

where δ represents the angle between the amino acid side chains; i represents the residue number in the position i from the sequence; Hi represents the ith amino acid's hydrophobicity on a hydrophobicity scale; Hxi represents the ith amino acid's helix propensity in Pace-Schols scale; and I represents the total number of residues present in the sequence.

In some embodiments, prior to step (b), the peptides in the population of parent peptides of (a) are subject to random crossing over between the peptides in the population.

In some embodiments, the amino acid sequence of at least one of the peptides in the population of parent peptides in (a) comprises the amino acid sequence of an antimicrobial peptide (AMP) or an AMP fragment. In some embodiments, the AMP or AMP fragment is a plant AMP or a plant AMP fragment. In some embodiments, the plant AMP or plant AMP fragment is Pg-AMP1 or a Pg-AMP1 fragment. In some embodiments, the Pg-AMP1 fragment is Pg-AMP1 fragment 2.

In some embodiments, the fraction of peptides selected from the population in (c) comprises at least 250 unique amino acid sequences. In some embodiments, the non-selected fraction of peptides in (c) comprise amino acid sequences corresponding to the 50 worst fitness values calculated in (b) or (e).

In some embodiments, at least one of the at least one property of interest is selected from the group consisting of α-helical propensity, higher net charge, hydrophobicity, and hydrophobic moment.

In some embodiments, the fitness function in (b) or (e) is represented by the equation:

$$Fitness = \frac{\sqrt[2]{{[ {\sum }_{i = 1}^{I} {H}_{i} \times cos ( δ i ) ]}^{2} + {[ {\sum }_{i = 1}^{I} {H}_{i} \times sin ( δ i ) ]}^{2}}}{{\sum }_{i = 1}^{I} {e}^{{Hx}_{i}}}$$

where δ represents the angle between the amino acid side chains; i represents the residue number in the position i from the sequence; Hi represents the ith amino acid's hydrophobicity on a hydrophobicity scale; Hxi represents the ith amino acid's helix propensity in Pace-Schols scale; and I represents the total number of residues present in the sequence.

In other aspects, the disclosure relates to antimicrobial peptides (AMPs).

In some embodiments, an AMP is designed according to the methods described herein. In some embodiments, the AMP has a minimal inhibitory concentration (MIC) that is lower than or equal to the peptide from which it was derived.

In some embodiments, an AMP comprises the amino acid sequence of any one of SEQ ID NOs: 1-100. In some embodiments, the AMP comprises the amino acid sequence RQYMRQIEQALRYGYRISRR (SEQ ID NO: 2) from N-terminal to C-terminal.

In other aspects, the disclosure relates to compositions comprising an AMP described herein. In some embodiments, a composition further comprises a pharmaceutically acceptable carrier and/or excipient.

In yet other aspects, the disclosure relates to methods of treating a patient having a bacterial infection comprising administering an AMP described herein or a composition described herein to the patient. In some embodiments, the bacterial infection is a gram-negative bacterial infection. In some embodiments, the gram-negative bacteria is selected from the group consisting of Escherichia coli, Pseudomonas aeruginosa, Klebsiella pneumonia, Acinetobacter baumanii, and Neisseria gonorrhoeae.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1E. Design and selection of artificially designed guavanins. FIG. 1A. Fragment mapping into the Pg-AMP1 sequence (SEQ ID NO: 106). Each fragment represents the maximum value of its respective physicochemical property: the α-helix propensity (0.553); the positive net charge (+3); the average hydrophobicity (−0.092); and the hydrophobic moment (0.3). FIG. 1B. Flowchart of the custom genetic algorithm. FIG. 1C. Fitness function evolution during the algorithm iterations (top to bottom on left side of graph: population; and best sequence). FIG. 1D. Amino acid distribution of guavanins and AMPs from APD2 and PhytAMP. Squares represent data obtained from 100 guavanin sequences; diamonds, the top 15 guavanins; down triangles, the overall APD2 composition; up triangles, the composition of α-helical peptides from APD2; and right triangles the plant AMP sequences from PhytAMP (Hammami et al., Nucleic Acids Res. 2008 Oct. 4; 37: D963-8). FIG. 1E. The frequency logo of the 100 generated guavanin sequences (TABLE 1), showing that they are arginine rich peptides, Arg residues are in at least 20% of their compositions.

FIGS. 2A-2B. Killing and membrane effects of lead synthetic peptide guavanin 2. FIG. 2A. Effect of guavanin 2 on plasma membrane integrity of E. coli ATCC 25922 cells after addition (vertical dotted line) of a concentration of peptide 2-fold above the MIC (12.5 μmol L⁻¹=32.8 μg mL⁻¹). The pore-forming peptide melittin (5 μmol L⁻¹=14.2 μg mL⁻¹) was used as a positive control. The negative control PBS corresponds to the bacteria incubated with the fluorescent probes without peptide. (Left) Time-course cytoplasmic membrane permeation analysis of SYTOX Green uptake. (Right) Cytoplasmic membrane hyperpolarization using DiSC3(5). FIG. 2B. SEM-FEG visualization of the effect of guavanin 2 on P. aeruginosa ATCC 27853. The control without peptide is displayed in the left panel. Bacteria were treated with a concentration of guavanin 2 corresponding to 25 μmol L⁻¹ (65.6 μg mL⁻¹—middle panel) and 50 μmol L⁻¹ (131.2 μg mL⁻¹—right panel), respectively. Scale bar=1 μm.

FIGS. 3A-3D. Structural analysis of guavanin 2. FIG. 3A. CD spectra of guavanin 2 at 25° C. and (33 μmol L⁻¹) in water pH 7.0; (38 μmol L⁻¹), pH 4.0, in DPC (20 mmol L⁻¹), SDS (20 mmol L⁻¹) and TFE/water (1:1, v:v) (top to bottom on left side of graph: DPC; SDS; TFE; and water). FIG. 3B. Solution NMR structure of guavanin 2 in 100 mM (DPC-d₃₈) micelles; A ribbon representation structure of lowest energy structure with side chains labeled. FIG. 3C. Ensemble of 10 backbone structures with low energy. FIG. 3D. Electrostatic surfaces of guavanin 2 in 100 mmol L⁻¹ (DPC-d₃₈) micelles. Surface potentials were set to ±5 kT e⁻¹ (133.56 mV). Charged residues are labeled.

FIGS. 4A-4B. In vivo activity of guavanin 2. FIG. 4A. Schematic of the experimental design. Briefly, the back of mice was shaved and an abrasion was generated to damage the stratum corneum and the upper layer of the epidermis. Subsequently, an aliquot of 50 μL containing 5×10⁷ CFU of P. aeruginosa in PBS was inoculated over each defined area. One day after the infection, peptides Pg-AMP1, guavanin 2, and Pg-AMP1 charge fragment were administered to the infected area. Animals were euthanized and the area of scarified skin was excised four (FIG. 4B) days post-infection, homogenized using a bead beater for 20 minutes (25 Hz), and serially diluted for CFU quantification. Two independent experiments were performed with 4 mice per group in each case. Statistical significance was assessed using a two-way ANOVA. At all doses tested treatment with guavanin 2 significantly reduced CFU counts (p<0.0001). Treatment with Pg-AMP1 and fragment 2 led to significant reduction of bacterial load only at higher concentrations (25 and 100 μg mL⁻¹).

FIG. 5. Sequence Alignment of guavanin 2 and the Pg-AMP1 fragments used as the initial population of the genetic algorithm. The residues inherited from each the fragments are highlighted and the mutated residues are in bold face. Guavanin 2—SEQ ID NO: 2; Fragment 1—SEQ ID NO: 101; Fragment 2—SEQ ID NO: 102; Fragment 3—SEQ ID NO: 103; Fragment 4—SEQ ID NO: 104.

FIG. 6. Ab initio models of the 4 fragments of Pg-AMP1 (Fragments 1-4) and the 15 guavanins with the best fitness values. Fragments 1 to 4 represent the best α-helical propensity, higher net charge, hydrophobicity and hydrophobic moment, respectively. Their physicochemical properties are detailed on TABLE 3. The four fragments present unusual predicted structures (Overall G-factors <−0.5). From guavanins, 13 out of 15 were predicted to be in 100% of α-helical structure. Guavanins 3 and 9 were predicted to have a loop in the C-terminal region, which is also considered unusual (Overall G-factors <−0.5). The model assessments are summarized in TABLE 5.

FIG. 7. Hydrogen bonding network involving side chains of guavanin 2. (Top) The N-Terminal region is stabilized by the residues Arg¹, Gln² and Tyr³, which interact with each other and whose positions vary depending on the structure evaluated from the NMR ensemble; the three possibilities observed are represented by structures 1, 2 and 10. (Bottom) The Gln⁹ side chain interacts with surrounding Arg residues (Arg⁵ and Arg¹²), the two possibilities observed are represented by structures 1 and 2.

FIG. 8. CD spectra of guavanin 2 at 25° C. in SDS (20 mmol L⁻¹) and pH 4.0, pH 7.0 and pH 10.0 (top to bottom on left side of graph: pH10; pH4; and pH7).

### DETAILED DESCRIPTION

AMPs are produced by virtually all living organisms on Earth as a defense mechanism. Plants are extensively used in traditional medicine and are also an excellent source of numerous natural products, including AMPs (Candido E. S. et al., (ed. Méndez-Vilas, A.) 951-960 (Formatex, 2011)). However, in more than 40 years of research, no plant AMP has been used to treat bacterial infections in humans, partly due to their limited antimicrobial activity and difficult synthesis using current methods of chemical synthesis (Harris et al., Chemistry. 2014 Mar. 8; 20(17): 5102-10; Cheneval et al., J. Org. Chem. 2014 Jun. 11; 79(12): 5538-44). Recent advancements in functional screening methods as well as improved strategies for peptide design hold promise in the development of novel AMP sequences with enhanced antimicrobial potency and/or with reduced length (Fjell et al., Nat. Rev. Drug Discov. 2011 Dec. 16; 11(1): 37-51; Porto et al., (ed. Faraggi, E.) 377-396 (InTech, 2012). doi: 10.5772/2335). Despite these advances, novel methods are needed for the cost-effective and rational design of innovative AMPs to translate these agents into the clinic.

There are two main approaches employed for the rational design of AMPs, in cerebro design and computer-aided design, both of which have been successfully used to generate novel AMP sequences (Diller et al., Future Med. Chem. 2015 Oct. 29; 7(16): 2173-93). However, both strategies are strongly influenced by the information encoded in AMP sequences deposited in databases, which limits their capacity to identify novel AMP sequences beyond those described in the literature. In cerebro design methods rely on the bacterial membrane as a target for AMPs. Because the bacterial membrane is hydrophobic and negatively charged, in practical terms, in cerebro design creates and/or modifies peptide sequences by means of increasing peptide cationicity and hydrophobicity, mainly by inserting lysine, isoleucine, and alanine residues within the sequence, thus enhancing the interaction between peptide and membrane (Thennarasu & Nagaraj, Protein Eng. 1996 December; 9(12): 1219-24; Cardoso et al., Sci. Rep. 2016 Feb. 26; 6: 21385). Computer-aided design methods, on the other hand, enable exploration of sequence space of AMPs using a number of algorithms. Unfortunately, and similar to in cerebro strategies, the optimal solutions obtained with such approaches end up sharing approximately 40% identity with AMP sequences deposited in the databases (Loose et al., Nature. 2006 Oct. 19; 443(7): 867-9; Maccari et al., PLoS Comput. Biol. 2013 Sep. 5; 9(9): e1003212; Porto et al., J. Theor. Biol. 2017 May 20; 426: 96-103), converging on a relatively small portion of AMP sequences composed of a restricted set of amino acids (Patel et al., J. Comput. Aided. Mol. Des. 1998 November; 12(6): 543-56; Fjell et al., Chem. Biol. Drug Des. 2010 Oct. 13; 77(1): 48-56). Even when incorporating non-proteinogenic amino acids into AMP sequences, for instance by exchanging ornithine or norleucine for cationic or hydrophobic residues, respectively (Maccari et al., PLoS Comput. Biol. 2013 Sep. 5; 9(9): e1003212; Giangaspero et al., Eur. J. Biochem. 2001 November; 268(21): 5589-600), this approach fails to identify novel AMP sequences with unique amino acid composition that may constitute novel drugs with enhanced antimicrobial potency.

Accordingly, disclosed herein are methods of designing peptides having at least one property of interest, such as α-helical propensity, higher net charge, hydrophobicity, and/or hydrophobic moment. Also disclosed herein are novel artificially evolved peptides (e.g., antimicrobial peptides), which may be designed according to the methods described herein, and methods of use thereof.

In some aspects, the disclosure relates to methods of designing peptides (e.g., antimicrobial peptides (“AMPs”)) having at least one property of interest (e.g., α-helical propensity, higher net charge, hydrophobicity, and/or hydrophobic moment). As used herein the term the term “peptide” refers to a sequence of three or more amino acids covalently attached through peptide bonds. The amino acid length of a peptide may vary. In some embodiments, a peptide comprises at least 10, at least 20, at least 30, at least 50, at least 100, or at least 500 amino acids.

In some embodiments, the method of designing peptides comprises: (a) selecting a population of parent peptides; (b) calculating a fitness function value for each peptide in the population of parent peptides of (a), wherein the fitness function value is indicative of the presence of at least one property of interest; (c) selecting a fraction of peptides from the population of peptides, wherein the fitness function values of the selected fraction of peptides are higher than the fitness function values of the non-selected fraction of peptides; (d) subjecting the fraction of peptides in (c) to fitness-guided mutation; (e) calculating a fitness function value for each peptide of (d), wherein the fitness function value is indicative of the presence of the at least one property of interest in (b); and (f) iteratively repeating steps (c)-(e).

The peptides in the population of parent peptides in (a) may be naturally-occurring or synthetic peptides (i.e., consisting of an amino acid sequence that is not found in nature). In some embodiments, each of the peptides in the population of parent peptides of (a) consists of a naturally occurring amino acid sequence. In other embodiments, each of the peptides in population of parent peptides in (a) consists of an artificial amino acid sequence. In yet other embodiments, the peptides in the population of parent peptides (a) comprise both naturally-occurring and artificial amino acid sequences.

In some embodiments, the population of parent peptides in (a) comprises peptides consisting of the same amino acid sequence. In other embodiments, the population of parent peptides in (a) comprises peptides comprising more than one amino acid sequence (i.e., the amino acid sequences of at least two peptides in the population of parent peptides differ). For example, in some embodiments, the population of parent peptides in (a) comprises two or more, three or more, four or more, five or more, six or more, seven or more, eight or more, nine or more, ten or more, twenty or more, thirty or more, forty or more, fifty or more, sixty or more, seventy or more, eighty or more, ninety or more, 100 or more, 150 or more, 200 or more, 250 or more, 500 or more, or 1000 or more unique amino acid sequences. Similarly, in some embodiments, the population of peptides in (a) comprises 2-5, 2-10, 2-20, 2-30, 2-40, 2-50, 2-60, 2-70, 2-80, 2-90, 2-100, 2-150, 2-200, 2-250, 2-500, 5-10, 5-20, 5-30, 5-40, 5-50, 5-60, 5-70, 5-80, 5-90, 5-100, 5-150, 5-200, 5-250, 5-500, 10-20, 10-30, 10-40, 10-50, 10-60, 10-70, 10-80, 10-90, 10-100, 10-150, 10-200, 10-250, 10-500, 20-30, 20-40, 20-50, 20-60, 20-70, 20-80, 20-90, 20-100, 20-150, 20-200, 20-250, 20-500, 50-60, 50-70, 50-80, 50-90, 50-100, 50-150, 50-200, 50-250, or 50-500 unique amino acid sequences.

In some embodiments, the peptides in the population of parent peptides in (a) are the same length. For example, in some embodiments each of the parent peptides is twenty amino acids in length. In other embodiments, the peptides in the population of parent peptides have varying lengths (i.e., at least two of the parent peptides have amino acid sequences that differ in length).

In some embodiments, each of the peptides in the population of parent peptides in (a) has essentially the same fitness function value. For example, in some embodiments, the peptides in the population of parent peptides have fitness values that differ by less than 10, less than 9%, less than 8%, less than 7%, less than 6%, less than 5%, less than 4%, less than 3%, less than 2%, less than 1%, or less than 0.5%. In some embodiments, each peptide in the population of parent peptides in (a) has the same fitness function value.

In some embodiments, the amino acid sequence of at least one of the peptides in the population of peptides of (a) comprises the amino acid sequence of an antimicrobial peptide (AMP). In some embodiments, the amino acid sequence of each of the peptides in the population of (a) comprises the amino acid sequence of an AMP. In some embodiments, the AMP is a naturally-occurring AMP. In other embodiments, the AMP is a synthetic AMP.

In plants, various AMPs with distinct composition have been identified, such as ones that are rich in glycine, histidine or proline residues (Pelegrini et al., Peptides. 2008 Mar. 22; 29(8): 1271-9; Park et al., Plant Mol. Biol. 2000 September; 44(2): 187-97; Cao et al., PLoS One. 2015 Sep. 18; 10(9): e0137414) the entireties of which are incorporated herein. Accordingly, in some embodiments, the AMP is produced in plants. In some embodiments, the plant AMP is Pg-AMP1. For example, the guava glycine-rich peptide Pg-AMP1 was used herein as a template to generate the novel “artificially designed” guavanin peptides by means of the methods described herein (see Examples 1-6).

In some embodiments, the AMP is produced naturally in an animal.

In some embodiments, the amino acid sequence of at least one of the peptides in the population of parent peptides of (a) comprises the amino acid sequence of an AMP fragment. As used herein, the term “AMP fragment” refers to a peptide comprising at least 8 amino acids of the AMP from which the fragment is derived. In some embodiments, the amino acid sequence of each of the peptides in the population of (a) comprises the amino acid sequence of an AMP fragment. In some embodiments, the AMP fragment is Pg-AMP1 fragment 2.

In some embodiments, prior to step (b), the peptides in the population of parent peptides in (a) are subject to random crossing over between the parent peptides in the population. The probability of change (i.e., probability of mutation) in the random crossing over may vary. For example, in some embodiments, the probability of mutation in an amino acid sequence (at one or more positions) may be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, or at least 5.0%. In some embodiments, the probability of mutation in an amino acid sequence (at one or more positions) may be 0.01%-0.05%, 0.01%-0.1%, 0.01%-0.2%, 0.01%-0.3%, 0.01%-0.4%, 0.01%-0.5%, 0.02%-0.05%, 0.02%-0.1%, 0.02%-0.2%, 0.02%-0.3%, 0.02%-0.4%, 0.02%-0.5%, 0.03%-0.05%, 0.03%-0.1%, 0.03%-0.2%, 0.03%-0.3%, 0.03%-0.4%, 0.03%-0.5%, 0.04%-0.05%, 0.04%-0.1%, 0.04%-0.2%, 0.04%-0.3%, 0.04%-0.4%, or 0.04%-0.5%. In some embodiments, the probability of mutation in an amino acid sequence (at one or more positions) in at least one iteration is 0.05%. In some embodiments, the random crossing over comprises a probability of a single-point cross over (i.e., a cross over occurring at one amino acid position within the amino acid sequence of each parent peptide). In other embodiments, the random crossing over comprises a probability of cross over between at least two, at least three, at least four, at least five, at least six, at least seven, at least 8, at least 9, or at least 10 amino acid positions within the amino acid sequence of each parent peptide.

The fraction of peptides selected in each iteration (i.e., step (c)) may vary. In some embodiments the fractions of peptides selected consists of less than 90%, less than 80%, less than 70%, less than 60%, less than 50%, less than 40%, less than 30%, less than 20%, or less than 10% of the total population of peptides. In some embodiments, the fraction of peptides selected in each iteration (i.e., step (c)) comprises ten or more, twenty or more, thirty or more, forty or more, fifty or more, sixty or more, seventy or more, eighty or more, ninety or more, 100 or more, 150 or more, 200 or more, 250 or more, 500 or more, or 1000 or more unique amino acid sequences. In some embodiments, the fraction of peptide selected in each iteration (i.e., step (c)) comprises 10-20, 10-30, 10-40, 10-50, 10-60, 10-70, 10-80, 10-90, 10-100, 10-150, 10-200, 10-250, 10-500, 20-30, 20-40, 20-50, 20-60, 20-70, 20-80, 20-90, 20-100, 20-150, 20-200, 20-250, 20-500, 50-60, 50-70, 50-80, 50-90, 50-100, 50-150, 50-200, 50-250, or 50-500 unique amino acid sequences. In some embodiments, the number of unique amino acid sequences selected in each iteration is the same. In other embodiments, the number of unique amino acid sequences selected in at least two iterations varies. In some embodiments, the number of unique amino acid sequences selected in each iteration varies.

In some embodiments, the non-selected fraction of peptides in (c) comprises amino acid sequences corresponding to at least the 10 worst fitness values, at least the 20 worst fitness values, at least the 30 worst fitness values, at least the 40 worst fitness values, at least the 50 worst fitness values, at least the 60 worst fitness values, at least the 70 worst fitness values, at least the 80 worst fitness values, at least the 90 worst fitness values, or at least the 100 worst fitness values calculated in (b) or (e). In some embodiments, the non-selected fraction of peptides in (c) comprises the amino acid sequences corresponding to the 50 worst fitness values calculated in (b) or (e).

The term “fitness-guided mutation” in step (d) refers to a process whereby the changes (i.e., mutations)—that are introduced into the amino acid sequences of the peptides in the fraction of peptides—are directed by a fitness function value. Changes may be introduced via any mechanism that alters the amino acid sequence of a peptide. For example, in some embodiments, a change may be introduced through at least one cross-over event with another peptide in the population of peptides. In some embodiments, a change may be introduced through at least one point mutation. In some embodiments, a change may be introduced through at least one cross-over event with another peptide in the population of peptides and at least one point mutation.

The probability of change (i.e., probability of mutation) in the fitness-guided mutation of (d) may vary. For example, in some embodiments, the probability of mutation in a unique amino acid sequence (at one or more positions) in at least one iteration may be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, or at least 5.0%. In some embodiments, the probability of mutation in a unique amino acid sequence (at one or more positions) in at least one iteration may be 0.01%-0.05%, 0.01%-0.1%, 0.01%-0.2%, 0.01%-0.3%, 0.01%-0.4%, 0.01%-0.5%, 0.02%-0.05%, 0.02%-0.1%, 0.02%-0.2%, 0.02%-0.3%, 0.02%-0.4%, 0.02%-0.5%, 0.03%-0.05%, 0.03%-0.1%, 0.03%-0.2%, 0.03%-0.3%, 0.03%-0.4%, 0.03%-0.5%, 0.04%-0.05%, 0.04%-0.1%, 0.04%-0.2%, 0.04%-0.3%, 0.04%-0.4%, or 0.04%-0.5%. In some embodiments, the probability of mutation in a unique amino acid sequence (at one or more positions) in at least one iteration is 0.05%.

In some embodiments, the fitness-guided mutation comprises a probability of a single-point cross over (i.e., a cross over occurring at one amino acid position within the amino acid sequence of each peptide in the fraction of peptides). In other embodiments, the fitness-guided crossing over comprises a probability of cross over between at least two, at least three, at least four, at least five, at least six, at least seven, at least 8, at least 9, or at least 10 amino acid positions within the amino acid sequence of each peptide in the population.

In some embodiments, at least one of the at least one property of interest is selected from the group consisting of, α-helical propensity, higher net charge, hydrophobicity, and hydrophobic moment. In some embodiments at least one of the at least one property of interest is α-helical propensity.

In some embodiments, a fitness function described herein is represented by the equation (i.e., a fitness value function is calculated from):

$$Fitness = \frac{\sqrt[2]{{[ {\sum }_{i = 1}^{I} {H}_{i} \times cos ( δ i ) ]}^{2} + {[ {\sum }_{i = 1}^{I} {H}_{i} \times sin ( δ i ) ]}^{2}}}{{\sum }_{i = 1}^{I} {e}^{{Hx}_{i}}}$$

where δ represents the angle between the amino acid side chains; i represents the residue number in the position i from the sequence; Hi represents the i^{th} amino acid's hydrophobicity on a hydrophobicity scale; Hxi represents the i^{th} amino acid's helix propensity in Pace-Schols scale; and I represents the total number of residues present in the sequence.

The number of iterations of the methods described herein may vary. In some embodiments, the method comprises at least 100, at least 200, at least 300, or at least 500 iterations.

In some embodiments, the number of iterations does not result in the plateauing of the average fitness function value of the population of selected peptides of (e). As used herein, the term “plateauing of the average fitness function” refers to changes in the average fitness value of a selected population of peptides. When a fitness function has plateaued, the average fitness values of the selected population of peptides in iteration n and iteration n+1 are statistically equivalent.

In some embodiments, the method of designing peptides having at least one property of interest comprises: (a) selecting a population of peptides; (b) calculating a fitness function value for each peptide in the population of peptides of (a), wherein the fitness function value is indicative of the presence of at least one property of interest; (c) selecting a fraction of the peptides from the population of peptides, wherein the fitness function values of the selected fraction of peptides are higher than the fitness function values of the non-selected fraction of peptides; (d) introducing at least one amino acid change in each peptide in the selected fraction of peptide sequences of (c); (e) calculating a fitness function value for each peptide sequence of (d), wherein the fitness function value is indicative of the presence of the at least one property of interest in (b); and (f) iteratively repeating steps (c)-(e), wherein the number of iterations does not result in the plateauing of the average fitness function values of the population of selected peptides of (e).

In other aspects, the disclosure relates to synthetic (i.e., non-natural) antimicrobial peptides (AMPs). In some embodiments, a synthetic AMP is designed according to the methods described above (see also Examples 1-7).

In some embodiments, the AMP comprises a sequence listed in TABLE 1 (e.g., any one of SEQ ID NOs: 1-100). In some embodiments, the antimicrobial peptide comprises the amino acid sequence RQYMRQIEQALRYGYRISRR (SEQ ID NO: 2) from N-terminal to C-terminal.

In yet other aspects, the disclosure relates to compositions comprising an AMP. In some embodiments each AMP in the composition comprises the same amino acid sequence. In other embodiments the composition comprises at least two, at least three, at least four, at least five, at least six, at least seven, at least eight, at least nine, or at least ten AMPs, each comprising a unique amino acid sequence.

In some embodiments, the composition comprising the AMP is a therapeutic composition. A therapeutic composition can include a pharmaceutically-acceptable carrier. Generally, for pharmaceutical use, the therapeutic may be formulated as a pharmaceutical preparation or composition comprising at least one active unit (i.e., an AMP) and at least one pharmaceutically acceptable carrier, diluent or excipient, and optionally one or more further pharmaceutically active compounds. Such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such administration forms may be solid, semi-solid or liquid, depending on the manner and route of administration. For example, formulations for oral administration may be provided with an enteric coating that will allow the formulation to resist the gastric environment and pass into the intestines. More generally, formulations for oral administration may be suitably formulated for delivery into any desired part of the gastrointestinal tract. In addition, suitable suppositories may be used for delivery into the gastrointestinal tract. Various pharmaceutically acceptable carriers, diluents and excipients useful in therapeutic compositions are known to the skilled person.

As used herein, the term “pharmaceutically-acceptable carrier” refers to one or more compatible solid or liquid filler, diluents or encapsulating substances which are suitable for administration to a human or other subject contemplated by the disclosure. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers (e.g., antioxidants), gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

In yet other aspects, the disclosure relates to methods of treating a patient having an infection. In some embodiments, the method comprises administering an AMP (described above) or a composition (described above) to the patient. Administration may be through any route known to one having ordinary skill in the art. For example, administration may be oral, parenteral (such as by intravenous, intramuscular or subcutaneous injection or intravenous infusion), or topical. In addition, administration may be by inhalation, by a skin patch, by an implant, by a suppository, etc.

In some embodiments, the infection is a fungal infection. In other embodiments, the infection is a bacterial infection. Examples of bacterial infections are known to those having skill in the art. In some embodiments, the bacteria causing the infection is a gram-negative bacteria (e.g., Escherichia coli, Pseudomonas aeruginosa, Klebsiella pneumonia, Acinetobacter baumanii, and Neisseria gonorrhoeae). In some embodiments, the bacteria causing the infection is a gram-positive bacteria (e.g., Staphylococcus aureus, Streptococcus pyogenes, Listeria ivanovii, or Enterococcus faecalis).

### EXAMPLES

### Example 1. Design and Screening of Computationally Evolved Guavanins

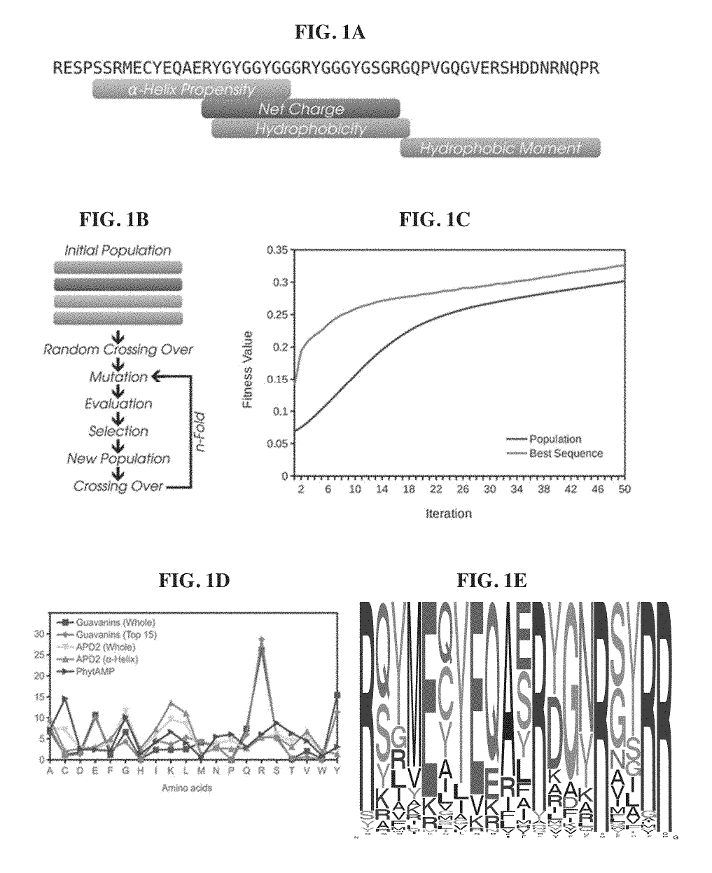

Overall, genetic algorithms (GAs) optimize a particular property (the fitness function) from a population of potential solutions (the sequences). Here, the hydrophobic moment and the α-helical propensity were used in the fitness function for selecting amphipathic α-helical peptides, while the initial population consisted of four Pg-AMP1 fragments derived according to specific physicochemical properties (FIG. 1A and FIG. 5). One hundred independent simulations of the algorithm were performed, with the parameters set as follows: 250 sequences in the population (generated by random crossing over in the first iteration and fitness guided crossing over in subsequent iterations), 50 with the worst fitness values for discard, single point cross over and 0.05% of probability of mutation—This mutation rate allows ˜6 mutations/sequence in the final population: 250 (sequences in the population) 50 (iterations) 0.05% (mutation rate) (FIG. 1B). As shown in FIG. 1C, the fitness values for the population and for the best sequence were improved without reaching stabilization, indicating a suboptimal solution.

The final set was composed of the best sequence of each parallel run, comprising peptides with fitness values varying from 0.245 to 0.393, named guavanins 1-100 (TABLE 1). The amino acid composition of all the guavanins is novel and different from other AMPs deposited in the Antimicrobial Peptides Database (APD), even taking into account only those peptides assigned with an α-helical structure (FIG. 1D); although guavanins are Arg-rich peptides, they contain Tyr residues as their hydrophobic counterpart (FIG. 1E).

TABLE 1
The best sequences of each parallel run of the genetic algorithm.

| Guavanin | | | Guavanin | | |
|---|---|---|---|---|---|
| and SEQ | | | and SEQ | | |
| ID NO | Sequence | Fitness | ID NO | Sequence | Fitness |
| 1 | RRGMKQYERISRDANRSYRR | 0.393 | 51 | RAYMECLEQAERYGNRAYRR | 0.324 |
| 2 | RQYMRQIEQALRYGYRISRR | 0.390 | 52 | RQVMETYEQLERYGNRSARR | 0.323 |
| 3 | RKYMRQYEEAIRDGNRSIRR | 0.390 | 53 | RQIRECYEQASRYGNRSYRR | 0.323 |
| 4 | RQYMRYLEQAERYVNRNLRR | 0.389 | 54 | RQYMEVYQEAERAGNRVYRR | 0.322 |
| 5 | RKLMEMYEEAFRYFNRISRR | 0.386 | 55 | RSYMEQYEQAFRRGNRSYRR | 0.322 |
| 6 | RSIMELYKQASRSFNRGIRR | 0.379 | 56 | RHFMECYEQASRDGNRSLRR | 0.321 |
| 7 | RQIYESIEQALRRGYRSYRR | 0.378 | 57 | RKAMEQYEEAERDGARSYRR | 0.321 |
| 8 | RSYYEAYERALRKGQRGIRR | 0.371 | 58 | RQYMKGYEQAERHAYRSYRR | 0.320 |
| 9 | RAYMEALRQAERLGNRTARR | 0.370 | 59 | RQYMEQAEQAERDGNRSVRR | 0.319 |
| 10 | RYLMEYAEQAKRDAKRAYRR | 0.370 | 60 | RSIMEYYEQIERDGNRSYRR | 0.318 |
| 11 | RQLMELIEQAERYGNRFYRR | 0.368 | 61 | RYLKECYEQASRIGYRGLRR | 0.318 |
| 12 | RKLMELYEQAIRYGKRSYRR | 0.364 | 62 | RQGMEAYEQAERLGNRGIRR | 0.318 |
| 13 | RRYMECYEQAERYFRRFGRR | 0.362 | 63 | RQYMECYKQIYRYGNRSYRR | 0.318 |
| 14 | RSFMKCYEQASRYGNRILRR | 0.362 | 64 | RSYREYAEQALRYGNRGYRR | 0.347 |
| 15 | RKLVECYERAERDANRSGRR | 0.361 | 65 | RSGMEYYKQAFRAGYRVTRR | 0.316 |
| 16 | RQLMECYEQAARRGARSYRR | 0.359 | 66 | RSAMECYEKAERYWYRGSRR | 0.316 |
| 17 | RYMMKIYEQAERYFNRVGRR | 0.359 | 67 | RSYMECYEQASRKGNRSIRR | 0.316 |
| 18 | RRYYEQLEQASRKGNRGFRR | 0.345 | 68 | RQYMELYQEAMRYGNRGYRR | 0.315 |
| 19 | RSVMEQYEQAARDAYRSARR | 0.355 | 69 | RQYIECYEQAARYGKRGYRR | 0.315 |
| 20 | RQYMECIEKALRDGYRSYRR | 0.352 | 70 | RQWAEYYEQLERYGNRSYRR | 0.315 |
| 21 | RYYMKCYKQAARYIYRGYRR | 0.351 | 71 | RSYMEAYEQASRDGYRLYRR | 0.314 |
| 22 | RSAYEYYRRAYRDGNRGYRR | 0.351 | 72 | RQYMEQYEQFERAGNRVYRR | 0.314 |
| 23 | RYGMRQFEQASRDGNRSFRR | 0.349 | 73 | RYYMEYYEKASRYGNRGIRR | 0.313 |
| 24 | RKGYRGYEQALRYGKRYGRR | 0.347 | 74 | RYYMEYYEQLERYGNRLYRR | 0.312 |
| 25 | RYGMRCLEEALRYGNRGYRR | 0.347 | 75 | RQYMECYEQAARYGNRSYRR | 0.309 |
| 26 | RQYREIIEAQRRVGNRGARR | 0.347 | 76 | RQYMEIYEQASRYGNRSYRR | 0.307 |
| 27 | RQGMEVYERASRQGNRSLRR | 0.346 | 77 | RQYMEQYEQAMRDGNRGYRR | 0.306 |
| 28 | RRIMEQYEEAERDGNRVYRR | 0.346 | 78 | RQYMEYYEQFSRLGNRSYRR | 0.305 |
| 29 | RQVMEAYEQFYRDGNRAYRR | 0.343 | 79 | RSGMKVYEQAERYGNRSYRR | 0.304 |
| 30 | RQLMEQYEQAYRYAARGYRR | 0.343 | 80 | RSAMECYEKASRDGNRGSRR | 0.304 |
| 31 | RYIMEIYEQAIRKGNRSYRR | 0.341 | 81 | RYYKEYYEKAERIGNRGYRR | 0.304 |
| 32 | RKYMELYEKASRRGYRGYRR | 0.338 | 82 | RSYMECYEQAFRYGKRSSRR | 0.303 |
| 33 | RQYLEQYENAERYIYRAYRR | 0.333 | 83 | RQYMECYKQAERYGNRGYRR | 0.302 |
| 34 | RQYMKCYEQAYRYGRRGYRR | 0.332 | 84 | RSVMEYYEQAYRYGNRGSRR | 0.301 |
| 35 | RQYAEQYEEAIRDGNRSVRR | 0.331 | 85 | RQGMEAYEQAERYGNRSYRR | 0.298 |
| 36 | RSYMEMLEQIERYGNRVGRR | 0.330 | 86 | RAYQEAYEQAYRDGNRSYRR | 0.298 |
| 37 | RQYMEFVEQAERYGRRGSRR | 0.330 | 87 | RSYMEQYEQASRKGYRSYRR | 0.298 |
| 38 | RSYMEQYEEAIRRGYRSYRR | 0.329 | 88 | RSYAECYEQISRYGNRGYRR | 0.298 |
| 39 | RQYMKYYEEAERYGNRAYRR | 0.328 | 89 | RSYMEAYEQAERYGNRGYRR | 0.296 |
| 40 | RAYMEYYEQFYRMGKRASRR | 0.328 | 90 | SQRVQEYVRRLYDDYRNYMR | 0.295 |
| 41 | RQYMEQVEQALRDGYRSGRR | 0.327 | 91 | RSYIEQYEQLERDGARSYRR | 0.294 |
| 42 | RSYMESIEQALRIGNRSYRR | 0.307 | 92 | SQRLERYVERSFDDYRKSGR | 0.292 |
| 43 | RSYMEIYEQASRAGNRAYRR | 0.327 | 93 | RSYMEYYEQASRDGARGYRR | 0.290 |
| 44 | RQYMEYYQEVFRAGYRSARR | 0.327 | 94 | SKRVGQGVERSYKKYRNYIR | 0.272 |
| 45 | RYYMECYEQAVRYGRRWYRR | 0.325 | 95 | GQRVEQLVERYGDDLRNSVR | 0.267 |
| 46 | RQGMECYEQALRYGQRGIRR | 0.325 | 96 | YQRVEQYVQRSYDAYRNYAR | 0.259 |
| 47 | RSFMEQGEQAFRDGYRMYRR | 0.325 | 97 | SQRVEQYVERYADGRYNYLR | 0.258 |
| 48 | RKYMEIYEKASRYGNRSYRR | 0.325 | 98 | YQRVEQYVQRYHDDLRNYSR | 0.256 |
| 49 | RQYKEAYEEIYRYGNRMGRR | 0.325 | 99 | YQRVEQYVQRSYDDYRNVGR | 0.245 |
| 50 | RRYMECYEQAERDGNRMYRR | 0.324 | 100 | TQRVEQYVERSSDKYRNLGR | 0.245 |

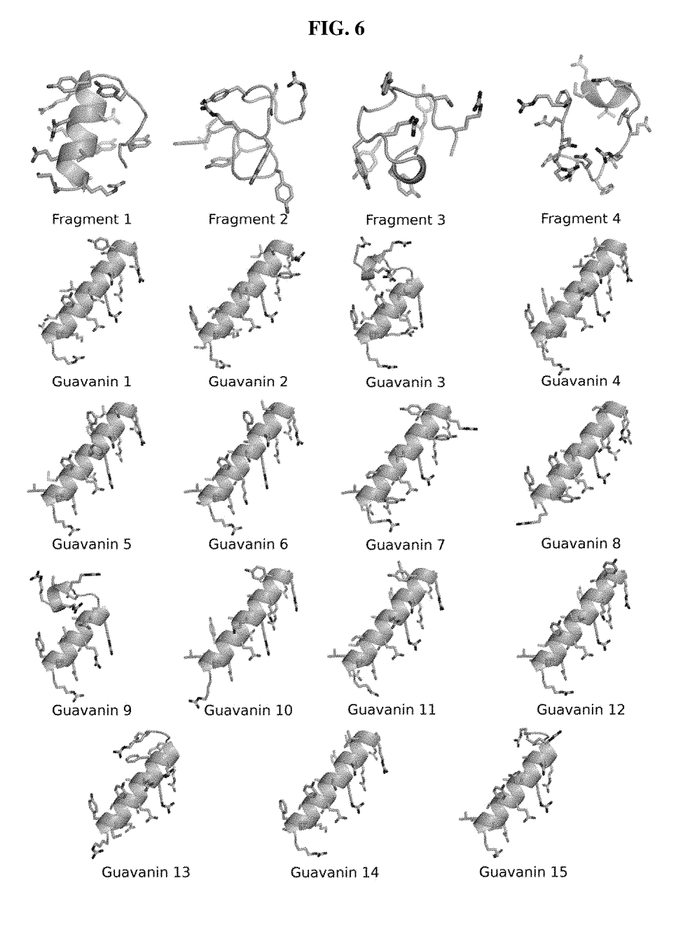

As the algorithm was interrupted prior to achieving an optimal solution (which would enrich for amino acids present in conventional AMPs), ab initio molecular modelling was then performed to verify the α-helical conformation for the 15 artificially generated guavanins with the greatest fitness value. All guavanins exhibited such structure (FIG. 6, TABLE 2), indicating that even in suboptimal solutions it is possible to obtain amphipathic α-helices, which is the basis of selection of the fitness function. As guavanins resembled AMPs, they were next synthesized chemically on cellulose membranes and screened for antimicrobial activity against P. aeruginosa and hemolytic activity using human erythrocytes (Winkler et al., Methods Mol. Biol. 2009; 570: 157-74).

TABLE 2
Structural assessments of ab initio models of the 4 Pg-AMP1
fragments and 15 best fitness guavanins

| | | | | Ramachandran | | |
|---|---|---|---|---|---|---|
| | SEQ | | | Plot (%) | | |
| | ID | | ProSA | Favored | Allowed | |
| Peptide | NO | DOPE | (Z-Score) | Regions | Regions | G-Factor |
| Fragment 1 ^{a} | 101 | −1228.738 | −1.22 | 100 | 0 | −0.99 |
| Fragment 2 ^{a,b} | 102 | −337.424 | −1.96 | 28.6 | 57.1 | −2.57 |
| Fragment 3 ^{a,b} | 103 | −489.954 | −1.27 | 71.4 | 14.3 | −2.26 |
| Fragment 4 ^{a,b} | 104 | −703.175 | −1.18 | 78.6 | 14.3 | −1.91 |
| Guavanin 1 | 1 | −1644.390 | −1.12 | 100 | 0 | −0.09 |
| Guavanin 2 | 2 | −1891.091 | −0.73 | 100 | 0 | −0.13 |
| Guavanin 3 ^{a} | 3 | −1519.247 | −0.99 | 94.1 | 5.9 | −0.80 |
| Guavanin 4 | 4 | −1950.491 | −1.25 | 100 | 0 | 0.10 |
| Guavanin 5 | 5 | −1902.878 | −1.00 | 100 | 0 | 0.01 |
| Guavanin 6 | 6 | −1633.499 | −0.48 | 100 | 0 | −0.26 |
| Guavanin 7 | 7 | −1779.689 | −1.08 | 100 | 0 | −0.17 |
| Guavanin 8 | 8 | −1563.839 | −1.14 | 100 | 0 | −0.28 |
| Guavanin 9 ^{a} | 9 | −1595.297 | −1.6 | 94.1 | 5.9 | −0.76 |
| Guavanin 10 | 10 | −1825.547 | −1.3 | 100 | 0 | 0.18 |
| Guavanin 11 | 11 | −1881.204 | −1.08 | 100 | 0 | 0.03 |
| Guavanin 12 | 12 | −1851.237 | −1.23 | 100 | 0 | −0.04 |
| Guavanin 13 | 13 | −1661.289 | −1.61 | 100 | 0 | −0.26 |
| Guavanin 14 | 14 | −1741.938 | −0.79 | 100 | 0 | −0.04 |
| Guavanin 15 | 15 | −1633.659 | −1.59 | 100 | 0 | −0.26 |

^{a} unusual structure according to G-Factor
^{b} Structures with at least five gly or pro residues, which are not taken into account for Ramachandran Plot analysis.

As shown in TABLE 3, 8 of the 15 guavanins analyzed were considered active because their MIC was lower than or equal to that of magainin 2 (100 μg mL⁻¹), the positive peptide control, and that of their parent peptide Pg-AMP1 (MIC of 100 μg mL⁻¹ vs P. aeruginosa). None of the peptides were hemolytic even at the highest concentration tested of 200 μg mL⁻¹ (TABLE 3). Interestingly, the determined MICs did not directly correlate in each case with the calculated fitness values (TABLE 3). As an example, guavanin 1 had the highest fitness value but the most potent peptide was the closely ranked guavanin 2 (TABLE 1). Therefore, while the fitness function employed here successfully identified novel AMPs, it did not systematically predict the antimicrobial potency of all the new sequences generated. However, the algorithm generated 4 hits (guavanin 2, 12, 13, and 14; TABLE 3).

TABLE 3
Physicochemical properites and biological activity assessment of
Pg-AMP1 fragments, guavanins 1-15 and magainin 2 (positive peptide control).

| | | | | | | | MIC | Hemolysis |
|---|---|---|---|---|---|---|---|---|
| Peptide | Sequence* | F | M | H | A | Q | (μg.mL⁻¹)** | (μg.mL⁻¹)*** |
| Fragment 1 | SSRMECYEQAERYGYG | n/a | 0.089 | −0.262 | 0.553 | 0 | >200 | >200 |
| (α-helix) | GYGG | | | | | | | |
| (SEQ ID NO: 101) | | | | | | | | |
| Fragment 2 | RYGYGGYGGGRYGGGY | n/a | 0.100 | −0.190 | 0.739 | +4 | 200 | 100 |
| (net charge) | GSGR | | | | | | | |
| (SEQ ID NO: 102) | | | | | | | | |
| Fragment 3 | YGYGGYGGRYGGGYGS | n/a | 0.027 | −0.092 | 0.779 | +3 | >200 | >200 |
| (hydrophobicity) | GRG | | | | | | | |
| (SEQ ID NO: 103) | | | | | | | | |
| Fragment 4 | GQPVGQGVERSHDDNR | n/a | 0.300 | −0.503 | 0.829 | +2 | >200 | >200 |
| (hydrophobic | NQPR | | | | | | | |
| moment) | | | | | | | | |
| (SEQ ID NO: 104) | | | | | | | | |
| Guavanin 1 | RRGMKQYERISRDANR | 0.393 | 0.589 | −0.773 | 0.379 | +7 | 200 | >200 |
| (SEQ ID NO: 1) | SYRR | | | | | | | |
| Guavanin 2 | RQYMRQIEQALRYGYR | 0.390 | 0.572 | −0.552 | 0.360 | +6 | 6.25 | >200 |
| (SEQ ID NO: 2) | ISRR | | | | | | | |
| Guavanin 3 | RKYMRQYEEAIRDGNR | 0.390 | 0.587 | −0.664 | 0.384 | +5 | >200 | >200 |
| (SEQ ID NO: 3) | SIRR | | | | | | | |
| Guavanin 4 | RQYMRYLEQAERYVNR | 0.389 | 0.560 | −0.627 | 0.350 | +5 | 100 | >200 |
| (SEQ ID NO: 4) | NLRR | | | | | | | |
| Guavanin 5 | RKLMEMYEEAFRYFNR | 0.386 | 0.552 | −0.479 | 0.345 | +4 | 100 | >200 |
| (SEQ ID NO: 5) | ISRR | | | | | | | |
| Guavanin 6 | RSIMELYKQASRSFNR | 0.379 | 0.568 | −0.477 | 0.380 | +6 | 100 | >200 |
| (SEQ ID NO: 6) | GIRR | | | | | | | |
| Guavanin 7 | RQIYESIEQALRRGYR | 0.378 | 0.562 | −0.574 | 0.373 | +5 | 200 | >200 |
| (SEQ ID NO: 7) | SYRR | | | | | | | |
| Guavanin 8 | RSYYEAYERALRKGQR | 0.371 | 0.558 | −0.598 | 0.371 | +6 | 100 | >200 |
| (SEQ ID NO: 8) | GIRR | | | | | | | |
| Guavanin 9 | RAYMEALRQAERLGNR | 0.370 | 0.516 | −0.553 | 0.298 | +5 | >200 | >200 |
| (SEQ ID NO: 9) | TARR | | | | | | | |
| Guavanin 10 | RYLMEYAEQAKRDAKR | 0.370 | 0.496 | −0.600 | 0.275 | +5 | 200 | >200 |
| (SEQ ID NO: 10) | AYRR | | | | | | | |
| Guavanin 11 | RQLMELIEQAERYGNR | 0.368 | 0.544 | −0.489 | 0.368 | +3 | >200 | >200 |
| (SEQ ID NO: 11) | FYRR | | | | | | | |
| Guavanin 12 | RKLMELYEQAIRYGKR | 0.364 | 0.526 | −0.544 | 0.346 | +6 | 25 | >200 |
| (SEQ ID NO: 12) | SYRR | | | | | | | |
| Guavanin 13 | RRYMECYEQAERYFRR | 0.362 | 0.545 | −0.658 | 0.383 | +5 | 25 | >200 |
| (SEQ ID NO: 13) | FGRR | | | | | | | |
| Guavanin 14 | RSFMKCYEQASFYGNR | 0.362 | 0.551 | −0.498 | 0.395 | +6 | 12.5 | >200 |
| (SEQ ID NO: 14) | ILRR | | | | | | | |
| Guavanin 15 | RKLVECYERAERDANR | 0.361 | 0.546 | −0.680 | 0.380 | +4 | 200 | >200 |
| (SEQ ID NO: 15) | SGRR | | | | | | | |
| Magainin 2 | GIGKFLHSAKKFGKAF | 0.168 | 0.286 | −0.036 | 0.489 | +5 | 100 | >200 |
| (SEQ ID NO: 105) | VGEIMNS | | | | | | | |

*All peptides were amidated in their Ct.
**MICs evaluated on SPOT-synthesized peptide samples of unpurified crude synthetic peptide
(~70% purity) against a bioluminescent engineered P. aeruginosa strain H1001.
***100% of hemolysis was not observed. F, fitness; μ, hydrophobic moment; H, hydrophobicity; α,
α-helix propensity; Q, net charge.

### Example 2. Guavanin 2 has a Narrow Spectrum of Activity Restricted to Gram-Negative Bacteria

Because guavanin 2 was the most potent peptide identified in the screening step (TABLE 3), it was selected for in depth analysis. Guavanin 2 was highly active against Gram-negative bacteria, particularly P. aeruginosa, Escherichia coli and Acinetobacter baumannii (TABLEs 3 and 4). Conversely, the peptide showed very modest or no killing activity towards Gram-positive bacteria (TABLE 4). The antifungal profile of guavanin 2 was also modest, exhibiting poor killing of the yeast Candida parapsilosis and was inactive against Candida albicans (TABLE 4).

TABLE 4
Antimicrobial activity and cytotoxicity
of synthetic peptide guavanin 2.

| | | Active |
|---|---|---|
| | | Concentration |
| Cell Strain | Microorganism/Cell Line | (μM)* |
| Gram-negative | Escherichia coli ATCC 25922 | 6.25 |
| bacteria | Pseudomonas aeruginosa ATCC 27853 | 25 |
| | Acinetobacter baumannii ATCC 19606 | 6.25 |
| Gram-positive | Staphylococcus aureus ATCC 25923 | 100 |
| bacteria | Streptococcus pyogenes ATCC 19615 | 50 |
| | Listeria ivanovii Li4pVS2 | 50 |
| | Enterococcus faecalis ATCC 29212 | >100 |
| Yeast | Candida albicans ATCC 90028 | >200 |
| | Candida parapsilosis ATCC 22019 | ≥50 |
| Human cells | Erythrocytes | >200 |
| | HEK-293 cells | >200 |

*The minimum inhibitory concentrations (MIC) for microorganisms, the lytic concentration 50 (LC₅₀) for erythrocytes, and the inhibitory concentration 50 (IC₅₀) for HEK-293 cells, are expressed as average values from three independent experiments performed in triplicate.

### Example 3. Guavanin 2 Exhibits a Safe In Vitro Selectivity Index for Gram-Negative Bacteria

In drug development, it is important that a drug candidate presents a safe therapeutic profile such that the amount of drug required to achieve a therapeutic effect is significantly lower than the amount that causes toxicity towards human cells. Here, the in vitro selectivity index of guavanin 2 was evaluated, which is analogous to the therapeutic index. Guavanin 2 toxicity for human erythrocytes and embryonic kidney cells (HEK-293) was investigated. Guavanin 2 displayed no detectable hemolytic activity (LC₅₀ higher than 200 μM) or cytotoxicity towards HEK-293 cells (IC₅₀ higher than 200 μM) (TABLE 4). Taking into account the MICs against Gram-negative bacteria and the cytotoxicity assessments, guavanin 2 showed a selectivity index of 23.93, indicating that to achieve a toxic effect, a fifteen-fold administration of this peptide would be necessary. Guavanin 2 is therefore almost five times safer than its recombinant predecessor Pg-AMP1, which has a selectivity index of 4.88 [based on data from Tavares et al., Peptides. 2012 Jul. 27; 37(2): 294-300]. In addition, the activity of guavanin 2 was tested against other eukaryotic cells to ensure the intended rational design (FIGS. 1A-1E) was selective towards bacterial cells. Consistent with the design principles, guavanin 2 exhibited poor killing of the yeast Candida parapsilosis and was inactive against Candida albicans (TABLE 4).

### Example 4. Guavanin 2 Kills Bacteria with Relatively Slow Membranolytic Kinetics

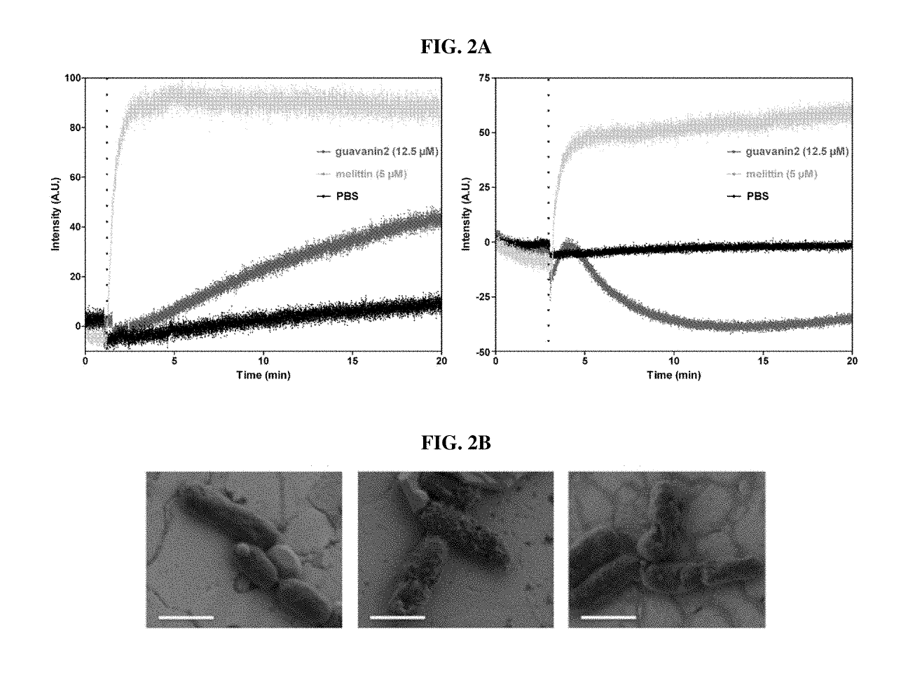

The killing kinetics of guavanin 2 against E. coli revealed that after 120 min of incubation at a peptide concentration of 12.5 μM (2-fold above the MIC), E. coli cells were reduced from 10⁷ to ˜10⁵ colony forming units, in contrast to the recently developed [I⁵, R⁸] mastoparan peptide that completely killed E. coli within 15 min (Irazazabal et al., Biochim. Biophys. Acta. 2016 Jul. 14; 1858(11): 2699-2708; Brogden, Nat. Rev. Microbiol. 2005 March; 3(3): 238-50). As the bacterial membrane is the main target of most AMPs, the membrane permeability and depolarization of E. coli cells was analyzed with SYTOX Green (SG) and DiSC3(5), respectively, with a peptide concentration identical to that used in the time-kill assays. As shown in FIG. 2A, a rapid and maximal SG fluorescence signal was reached after incubation of bacteria with 5 μM of melittin, a 26-residue AMP from bee venom that acts on bacterial membranes via pore formation and serves as a positive control for peptide-induced membrane damage (Rex, Biophys. Chem. 1996 Jan. 16; 58(1-2): 75-85). In contrast, guavanin 2 caused only a slow and very small amount of dye influx in comparison to the positive and negative controls. Surprisingly, a decrease in DiSC3(5) fluorescence was observed after incubating E. coli cells with guavanin 2 (FIG. 2A), suggesting that this peptide induces hyperpolarization of the bacterial membrane, unlike melittin (and numerous other AMPs), which produced a rapid increase in the fluorescence signal. Thus, guavanin 2, unlike most other AMPs, acts by hyperpolarizing the bacterial membrane. In order to obtain more insight into the killing mechanism of guavanin 2, a complementary SEM-FEG analysis of the Gram-negative bacterium P. aeruginosa ATCC 27853 was performed. SEM-FEG images clearly show membrane damage (deformations or indentations) of P. aeruginosa cells after incubation with 25 μM (MIC) and 50 μM of guavanin 2, in comparison to intact bacteria (FIG. 2B).

### Example 5. Guavanin 2 Undergoes a Coil-to-Helix Transition in Hydrophobic Environments

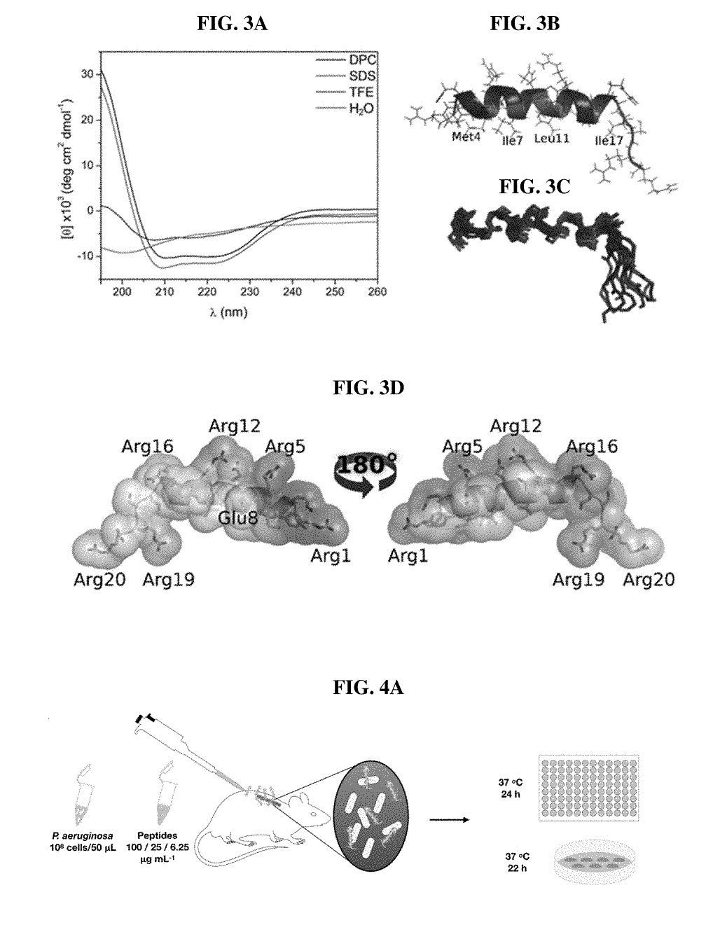

Ab initio molecular modelling was performed to verify the α-helical conformation of guavanins 1-15 (FIG. 6). These experiments confirmed that all peptides displayed an α-helical structure (FIG. 8). Guavanin 2 was used as a prototype “artificial” peptide for further in vitro structural analysis. As the target of guavanin 2 is the bacterial membrane (FIGS. 2A-2B), structural analysis was performed to verify that there was a conformational change in guavanin 2 when present in hydrophobic environments, and also to evaluate whether the fitness function of the GA generates a peptide capable of adopting an α-helical structure. Circular dichroism (CD) experiments of guavanin 2 in water (pH 7.0) indicated no defined secondary structure (FIG. 3A). At the same pH, an α-helical conformation was observed in SDS micelles (FIG. 8), indicating a coil-to-helix transition of guavanin 2 upon interaction with hydrophobic environments. The pH influence on the structure was also tested in SDS micelles, showing that guavanin 2 maintained an α-helical structure at pH 4.0, 7.0, and 10.0, and at pH 4.0 the peptide displayed the highest abundance of secondary structure (FIG. 8). To determine the best environment for NMR experiments, guavanin 2 was tested in SDS, DPC, and TFE. In SDS and DPC micelles (20 mmol L⁻¹) at pH 4.0, the peptide showed the highest abundance of secondary structure, presenting 42% and 39% of α-helical content, respectively (FIG. 3A).

The three-dimensional structure of guavanin 2 in the presence of deuterated dodecyl-phosphocoline (DPC-d₃₈) micelles, which are routinely used as a membrane mimetic (Wang, Biochim. Biophys. Acta. 2007 December; 1768(12): 3271-81; Usachev et al., J. Biomol. NMR. 2014 Nov. 28; 61(3-4): 227-34), was elucidated by using 2D NMR spectroscopy, and the structural statistics for 10 structures with low energy are summarized in TABLE 5. ¹H-¹H NOESY spectra revealed a total of 358 distance restraints with 17.9 average restrictions per residue. Guavanin 2 adopted an α-helical structure between residues Gln²-Arg¹⁶ in 100 mmol L⁻¹ of DPC-d₃₈ micelles, supporting the ab initio predictions (FIG. 6). The structure is highly precise, with a backbone RMSD of 0.88±0.25 Å over residues 2-16. Despite the random character of the C-terminal region, the heavy atoms RMSD, equivalent to 2.28±0.33, revealed that the structures were well defined and concise in DPC-d₃₈ micelles. Intra-side chain interactions also contributed to the defined geometry of the peptide. The residues Arg¹, Gln² and Tyr³ are involved in a hydrogen bonding network that stabilizes the N-terminal region; while Gln⁹ interacts with Arg⁵ or Arg¹², stabilizing the center of the structure. Guavanin 2 forms a relatively well ordered apolar cluster with aliphatic residues Met⁴, Ile⁷, Leu¹¹, and Ile¹⁷ (FIG. 3B). Thus, the existence of converging conformations showed regularity and agreement among the restraints used in the structural calculation (FIG. 3C). The electrostatic potential on the surface of the peptide structure revealed that guavanin 2 is highly cationic, suppressing the negative charge of Glu⁸ (FIG. 3D). Depending on the N-terminal protonation, the net charge of guavanin 2 varies from +5 to +6, as the C-terminal is amidated. The six arginine residues distributed along the structure neutralized the negative charge of Glu⁸, and generated a solvation potential energy of 2.38±0.33 MJ mol⁻¹. This net charge likely promotes the attraction of guavanin 2 to cell membranes composed of phospholipids with negatively charged head groups, which is considered the first stage of its mechanism of action towards Gram-negative cells.

TABLE 5
NMR structural statistics for the 20 lowest-
energy structures of guavanin 2.

| Structural Assessment | Parameter | Value |
|---|---|---|
| NOE distance restrains | Intraresidue | 204 |
| | Sequential | 116 |
| | Medium range (1 ≤ \|I − j\| ≤ 5) | 38 |
| | Long range (\|I − j\| > 5) | 0 |
| | Total | 358 |
| TALOS+ | Dihedral angle restraints | 36 |
| | Average restrictions per residue | 17.9 |
| RMSD (Å) ^{b} | Heavy atoms (residues 1-20) | 2.28 ± 0.33 |
| | Backbone atoms (residues 1-20) | 1.37 ± 0.34 |
| | Heavy atoms (residues 2-16) | 1.86 ± 0.24 |
| | Backbone atoms (residues 2-16) | 0.88 ± 0.25 |
| Ramachandran plot^{c} | Favored regions | 100% |
| G-Factors^{c} | Phi-psi distribution | 0.17 ± 0.08 |
| | Chi1-chi2 distribution | −1.78 ± 0.20 |
| | Chi1 only | −0.24 ± 0.66 |
| | Chi3 and chi4 | 0.55 ± 0.14 |
| | Omega | 0.58 ± 0.06 |
| | Average | −0.10 ± 0.07 |
| | Main-chain bond lengths | 0.61 ± 0.01 |
| | Main-chain angles | 0.55 ± 0.02 |
| | Average | 0.57 ± 0.01 |
| | Overal average | 0.14 ± 0.04 |
| ProSA | Z-Score | 0.07 ± 0.4 |

^{a} Predicted by TALOS+.
^{b} Calculated by MOLMOL.
^{c}Calcualted by PROCHECK.

### Example 6. Guavanin 2 Exhibits Anti-Infective Potential in a Murine Abscess Skin Infection Model

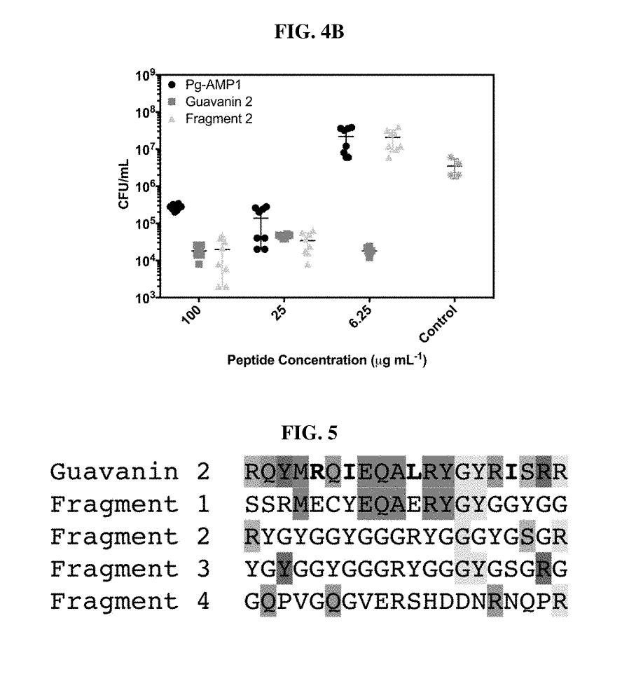

In order to test the activity of guavanin 2 in a clinically relevant animal model (FIG. 4A) and compare its anti-infective activity to that of its parent peptides Pg-AMP1 and Pg-AMP1 fragment 2, an established abscess skin infection mouse model was leveraged (FIGS. 4A-4B). Mice were infected with P. aeruginosa, and a single dose of peptides was administered to the site of infection 24 hours later. Treatment with guavanin 2 led to a 3-log reduction in bacterial counts after 4 days, even at the lowest dose tested of 6.25 kg mL⁻¹ (FIG. 4B). On the other hand, naturally occurring wild-type peptide Pg-AMP1 and the Pg-AMP1 fragment 2 derivative exhibited no activity at 6.25 μg mL⁻¹ (FIG. 4B). All peptides displayed comparable anti-infective activity at higher concentrations (25 and 100 μg mL⁻¹) (FIG. 4B).

### Example 7. Materials and Methods for Examples 1-6

Genetic Algorithm (GA): The GA simulates the evolution of a population of sequences during n iterations, where given iteration Iₙ generates the population Pₙ from the population Pₙ₋₁, evaluating the sequences according to the value of a fitness function, also known as “chance of survivor and mating” (FIGS. 1A-1E). The fitness function was given by equation 1. The algorithm was implemented in PERL. In the first iteration (I₁) of the implementation of the custom GA, all sequences from P₀ had the same fitness value, thus providing a random selection for each sequence pair (FIGS. 1A-1E). From iteration 12 to Iₙ, the sequence selection for mating was performed according the corresponding fitness values. For each iteration, 250 sequence pairs were selected from population Pₙ and each pair was submitted to a crossing over process, generating a new sequence pair for population Pₙ₊₁. Each novel sequence had a 0.05% chance of mutation, where one residue was randomly selected for substitution. The replacement was chosen according to the probability distribution listed in TABLE 6. From the replacing residues list, Gly and Pro were removed due to poor α-helix formation; Asp and Glu due to their negative charge; and Cys due to the possibility to form disulfide bridges. After that, the sequences from Pₙ₊₁ were evaluated by the fitness function and were subsequently ranked. The 50 worst sequences were removed from the population Pₙ₊₁ and then a novel iteration step began (FIG. 1B). The cycle was repeated until the number of iterations was exhausted. For the development of synthetic guavanins, 100 independent simulations were performed, each one with 50 iterations using the same conditions. The best sequence of each independent simulation was chosen and then ranked; the 15 best sequences according to the fitness function were selected for further evaluation.

TABLE 6
Amino acid probability distributions. This distribution was
based on the frequency of occurrence of each amino acid according
to the Antimicrobial Peptides Database (APD - Accessed on April,
2013. Cysteine, aspartic acid, glutamic acid, glycine and proline
residues were removed from the set and the probability distribution
was adjusted for remaining residues.

| Residue | Distribution (%) |
|---|---|
| A | 11.092 |
| F | 5.624 |
| H | 2.925 |
| I | 8.563 |
| K | 13.494 |
| L | 11.869 |
| M | 1.597 |
| N | 5.341 |
| Q | 3.207 |
| R | 7.984 |
| S | 8.281 |
| T | 6.132 |
| V | 8.111 |
| W | 2.247 |
| Y | 3.533 |

Fitness Function: The equation 1 was designed to generate amphipathic α-helical peptides, based on the ratio between Eisenberg's hydrophobic moment and the sum of exponential α-helix propensity in Pace-Schols scale:

$$Fitness = \frac{\sqrt[2]{{[ {\sum }_{i = 1}^{I} {H}_{i} \times cos ( δ i ) ]}^{2} + {[ {\sum }_{i = 1}^{I} {H}_{i} \times sin ( δ i ) ]}^{2}}}{{\sum }_{i = 1}^{I} {e}^{{Hx}_{i}}} \qquad ( 1 )$$

Where δ represents the angle between the amino acid side chains (100° for α-helix, on average); i, the residue number in the position i from the sequence; Hi, the i^{th} amino acid's hydrophobicity on a hydrophobicity scale; Hxi, the i^{th} amino acid's helix propensity in Pace-Schols scale (Pace et al., Biophys. J. 1998 July; 75(1): 422-427); and I, the total number of residues present in the sequence.

Instead of directly using the hydrophobic moment equation, modifications were introduced into the equation to account for α-helix propensity, because it was observed that in Pg-AMP1, the C-terminal portion showed the highest hydrophobic moment (FIG. 1A and TABLE 3), but in previous studies this portion was intrinsically unstructured (Pelegrini et al., Peptides. 2008 Mar. 22; 29(8): 1271-9; Porto et al., Peptides. 2014 Feb. 26; 55: 92-7). Therefore, the hydrophobic moment per se does not guarantee α-helix formation. As the Pace-Schols α-helix propensity is given in terms of the amount of energy required for a given amino acid residue to adopt an α-helical conformation (i.e. the lower energy, the easier for that residue to adopt an α-helical conformation), the α-helix propensity was introduced in the denominator of Equation 1. However, using the α-helix propensity in the denominator has a bias: as the scale is normalized by subtracting the resulting values from that of alanine, thus, the normalized value of alanine is zero. Therefore, the algorithm tends to lower the value of α-helix propensity because it is in the denominator. However, if α-helix propensity reaches a zero value, it would generate a division by zero (formally a/0=∞, being “a” a positive number), hindering the algorithm progress. Therefore, by using the exponential values of Pace-Schols scale, one could avoid the division by zero (as e⁰=1).

Computational Selection of Pg-AMP1 Fragments: In order to identify regions of Pg-AMP1 with potential antimicrobial activity, the Pg-AMP1 sequence was submitted to a sliding window system, selecting windows of 20 amino acid residues and generating 36 fragments. For each fragment, four independent properties were calculated: α-helix propensity, positive net charge, hydrophobicity and hydrophobic moment. For each property, one fragment was selected (FIG. 5 and TABLE 3). The α-helix propensity was calculated by using the α-helix propensity scale from Pace and Scholtz (Pace et al., Biophys. J. 1998 July; 75(1): 422-427) and the hydrophobicity and hydrophobic moment were measured using the Eisenberg's hydrophobic scale (Eisenberg et al., Faraday Symp. Chem. Soc. 1982; 17, 109). The hydrophobic moment was calculated using Eisenberg's equation (Eisenberg et al., Faraday Symp. Chem. Soc. 1982; 17, 109). The composition of guavanins was compared with APD2 (Wang et al., Nucleic Acids Res. 2008 Oct. 28; 37: D933-7), for general and α-helix peptides; and PhytAMP for plant peptides (Hammami et al., Nucleic Acids Res. 2008 Oct. 4; 37: D963-8).

Ab Initio Molecular Modelling:

QUARK ab initio modelling server was used for generating the three-dimensional models of the 4 Pg-AMP1 fragments and the 15 best fitness guavanins. The models were evaluated through, ProSA II and PROCHECK (Xu & Zhang, Proteins. 2012 Apr. 13; 80(7): 1715-35; Wiederstein & Sippl, Nucleic Acids Res. 2007 May 21; 35: W407-10; Laskowski et al., PROCHECK: a program to check the stereochemical quality of protein structures. J. Appl. Cryst. 1993; 26: 283-291). PROCHECK checks the stereochemical quality of a protein structure, through the Ramachandran plot, where reliable models are expected to have more than 90% of amino acid residues in most favored and additional allowed regions. PROCHECK also gives the G-factor, a measurement of how unusual the model is, where values below −0.5 are unusual, while PROSA II indicates the fold quality. The MODELLER 9.17 build in function for the discrete optimized protein energy score (DOPE score) was also used to assess the models (Webb & Sali, Curr. Protoc. Bioinformatics. 2014 Sep. 8; 47: 5.6.1-5.6.32).

High-Throughput Peptide Synthesis on Cellulose Arrays:

A peptide array composed of 20 peptides (15 guavanins, 4 Pg-AMP1 fragments and magainin 2) was designed and synthesized by Kinexus Bioinformatics Corporation (Vancouver, BC). Peptides were produced in a standard mass of 80 μg by using cellulose support in SPOT technology, as previously described by Winkler et al. Methods Mol. Biol. 2009; 570: 157-74. The crude synthetic peptides were obtained from cellulose membrane discs that had already been treated with ammonia gas to release the peptides from the membrane. Peptides were then dissolved overnight in distilled water and subsequently evaluated for their biological activities, as described below.

Determination of Antimocrobial Activity by Bioluminescence Assays:

The antimicrobial activity of the synthesized peptides was evaluated against an engineered luminescent Pseudomonas aeruginosa H1001 strain in 96-well microplates, as described previously with a few modifications (Hilpert & Hancock, Nat. Protoc. 2007; 2(7): 1652-60). Aqueous solutions of peptides released from the cellulose spots were diluted two-fold in BM2 medium [62 mM potassium phosphate buffer pH 7; 2 mM MgSO₄; 10 μM FeSO₄; 0.4% (wt/vol) glucose] down the 8 wells of a 96 well plate, achieving a final volume of 25 μL in each well. Subsequently, 50 μL of overnight culture of P. aeruginosa H1001 (fliC::luxCDABE) were subcultured in 5 mL of fresh LB media and grown until they reached an OD₆₀₀ of 0.4. This growing bacteria culture was then diluted 4:100 (v/v) into fresh BM2 media and 25 μL of this diluted bacterial culture was transferred to the microplate wells containing 25 μL of peptide solution. The final peptide concentrations tested ranged from 200 to 3 μg·mL⁻¹. The plates were incubated for 4 h at 37° C. with constant shaking at 50 rpm. Luminescence was measured on a Tecan SPECTRAFluor Plus Microplate Reader (Tecan US, Morrisville, N.C.). The antimicrobial activity was evaluated by the ability of the peptides to reduce the luminescence of P. aeruginosa-lux strain compared to untreated cells. The AMP magainin 2 and the carbapenem meropenem were used as positive controls and distilled water was used as a negative control.

Hemolytic Assays:

Fresh human venous blood was collected from volunteers in Vacutainer collection tubes containing sodium heparin as an anticoagulant (BD Biosciences, Franklin Lakes, N.J.). The blood was centrifuged at 1500 rpm and the serum was removed and the blood cells were replaced and washed 3 times with the same volume of sterile NaCl 0.85% solution. Concentrated red blood cells were diluted tenfold in NaCl 0.85% solution and then exposed at two-fold dilutions of peptides for 1 h at 37° C., at identical concentrations used for antimicrobial assays, in the ratio of 1:1 (v/v), achieving a final volume of 100 uL. The assay was carried out in 96-well polypropylene microtiter plates. The positive control wells contained 1% of Triton X-100, representing 100% cell lysis, and negative control wells contained sterile saline. Hemoglobin release was monitored chromogenically at 546 nm using a microplate reader.

Peptide Synthesis by Solid-Phase:

The peptide guavanin 2 was synthesized by stepwise solid-phase using the N-9-fluorenylmethyloxycarbonyl (FMOC) strategy and purified by high-performance liquid chromatography (HPLC), with purity >95% by Peptide 2.0 (Virginia, USA). The sequence and degree of purity (>95%) was confirmed by MALDI-ToF analyses (Cardoso et al., Sci. Rep. 2016 Feb. 26; 6: 21385).

Antimicrobial Activity: The minimal inhibitory concentration (MIC) of guavanin 2 was determined in 96-well microtitre plates by growing the microorganisms in the presence of two-fold serial dilutions of the peptide, as previously described (Abbassi et al., Peptides. 2008 September; 29(9): 1526-33). Staphylococcus aureus ATCC 25923, Enterococcus faecalis ATCC 29212, Escherichia coli ATCC 25922, Pseudomonas aeruginosa ATCC 27853, Acinetobacter baumannii ATCC 19606 and Klebsiella pneumoniae ATCC 13883 were cultured in Lysogeny Broth (LB). The bacteria Streptococcus pyogenes ATCC 19615 and Listeria ivanovii Li 4pVS2 were cultured in Brain Heart Infusion (BHI) broth, whereas Candida species (C. albicans ATCC 90028 and C. parapsilosis ATCC 22019) were cultured in Yeast Peptone Dextrose (YPD) medium. Logarithmic phase culture of bacteria and yeasts were centrifuged and suspended in MH (Mueller Hinton) broth to an A₆₃₀ of 0.01 (˜10⁶ CFU·mL⁻¹), except for S. pyogenes, L. ivanovii and E. faecalis that were suspended in their respective growth medium. 50 μL of the microorganism suspension was mixed with 50 μL of guavanin 2 at different concentrations (200 to 1 μM, final concentrations). After 18 h incubation at 37° C. (30° C. for yeasts), the antimicrobial susceptibility was monitored by measuring the change in A₆₃₀ using a microplate reader (UVM 340, Asys Hitech). The MIC was determined as the lowest peptide concentration that completely inhibited the growth of the microorganism and corresponds to the average value obtained from three independent experiments. Each experiment was performed in triplicate with positive (0.7% formaldehyde) and negative (without peptide) inhibition controls.

Cytoxic Profiles:

The cytotoxicity of guavanin 2 was determined against the human embryonic kidney cell line HEK-293. HEK-293 cells were cultured in DMEM medium, and incubated at 37° C. in a humidified atmosphere of 5% CO₂. Cell viability was quantified after peptide incubation using a methylthiazolyldiphenyl-tetrazolium bromide (MTT)-based microassay (Riss et al., (eds. Sittampalam, G. et al.) (Bethesda (Md.), 2004)). Briefly, cells were seeded on 96-well culture plates at a density of 5×10⁵ cells·mL⁻¹ and incubated 72 h at 37° C. with 100 μl of guavanin 2 at different concentrations (12.5 to 200 μM, final concentrations). Then, 10 μl of MTT (5 mg·mL⁻¹ in PBS) was added to each well and the cells were further incubated for 4 h in the dark. The formazan crystals formed by mitochondrial reductases in intact cells are insoluble in aqueous solutions and precipitate. Formazan crystals were dissolved using a solubilization solution (40% dimethylformamide in 2% glacial acetic acid, 16% sodium dodecyl sulfate, pH 4.7) followed by 1 h incubation at 37° C. under shaking (150 rpm). Finally, the absorbance of the resuspended formazan was measured at 570 nm. Data were analyzed with GraphPad Prism® 5.0 software to determine the inhibitory concentration 50 (IC₅₀), which corresponds to the peptide concentration producing 50% cell death. Results were expressed as the mean of three independent experiments performed in triplicate.

In Vitro Selectivity Index Calculation:

The in vitro selectivity index is analogous to the therapeutic index concept, corresponding to the ratio between cytotoxic effect and antibacterial effect. The selectivity index of guavanin 2 was calculated according to Chen et al. (53) with minor modifications, using equation 2:

$$SI = \frac{\sqrt[n]{{\prod }_{i = 1}^{n} {Cytotoxic}_{i}}}{\sqrt[m]{{\prod }_{j = 1}^{m} {Antibacterial}_{j}}} \qquad ( 2 )$$

Where n is the number of cytotoxic assays with different cells and m is the number of antimicrobial assays with different bacteria. For values higher than the maximum concentration tested, it was assumed twofold the maximum tested value (e.g. if the value is higher than 100, it was considered as 200) (Chen et al., J. Biol. Chem. 2005 Apr. 1; 280(13): 12316-29).

Time-Kill Studies:

The killing kinetics of guavanin 2 against the Gram-negative bacterium E. coli ATCC 25922, were investigated as previously described (Pelegrini et al., Peptides. 2008 Mar. 22; 29(8): 1271-9). Exponentially growing bacteria in LB were harvested by centrifugation, washed three times in PBS and suspended in the same buffer to a final concentration of 10⁶ CFU·mL⁻¹. 100 μL of this bacterial suspension was incubated with a dose of peptide corresponding to two-fold the MIC. Then, aliquots of 10 μL were withdrawn at different times, diluted in LB, and spread onto LB agar plates. The CFU were counted after overnight incubation at 37° C. Two experiments were carried out in triplicate and controls were run without peptide.

SYTOX Green Uptake Assay:

The guavanin 2-induced permeabilization of the bacterial cytoplasmic plasma membrane of E. coli ATCC 25922 was determined by fluorometric measurement of SYTOX green (SG) influx (Thevissen et al., Appl. Environ. Microbiol. 1999 December; 65(12): 5451-8). SG is a high-affinity nucleic acid dye that is impermeant to live cells. When the cell membrane is damaged, this dye penetrates into the cell and binds to intracellular DNA, leading to an increase in fluorescence. For SG uptake assay, exponentially growing bacteria (6×10⁵ CFU·mL⁻¹) were re-suspended in PBS after centrifugation (1000×g, 10 min, 4° C.) and washing steps. 792 μL of the bacterial suspension was pre-incubated with 8 μL of 100 μM SG during 30 min at 37° C. in the dark. After peptide addition (200 μL, final concentration two-fold above the MIC), a Varian Cary Eclipse fluorescence spectrophotometer was used to monitored the fluorescence for 1 h at 37° C., with excitation and emission wavelengths of 485 and 520 nm, respectively. Three independent experiments were performed and results correspond to a representative experiment with negative (PBS) and positive (melittin) controls.

Membrane Polarization Assay:

To study the ability of guavanin 2 to alter the plasma membrane potential, the membrane depolarization of E. coli (ATCC 25922) was evaluated using the membrane potential-sensitive fluorescent probe DiSC3(5) (3,3′-dipropylthiadicarbocyanine iodide) (Sims et al., Biochemistry. 1974 Jul. 30; 13(16): 3315-30). When the cytoplasmic membrane is intact, the fluorescent probe DiSC3(5) accumulates into the cytoplasmic membrane and then aggregates, causing self-quenching of the fluorescence. In the presence of a membrane-depolarizing agent, DiSC3(5) is released into the medium, leading to an increase in fluorescence that can be monitored over time. The experiment was performed as previously described (André et al., ACS Chem. Biol. 2015 Jul. 30; 10(10): 2257-66). Briefly, exponentially growing bacteria were centrifuged (1000×g, 10 min, 4° C.), washed with PBS and re-suspended in the same buffer to an A₆₃₀ of 0.1; then 700 L of bacteria were pre-incubated with 1 μM DiSC3(5) in the dark during 10 min at 37° C., and then 100 μL of 1 mM KCl were added in order to equilibrate the cytoplasmic and external K+ concentrations. After addition of guavanin 2 (200 μL, final concentration: two-fold above the MIC), the changes in fluorescence were recorded at 37° C. for 20 min at an excitation wavelength of 622 nm and an emission wavelength of 670 nm (Varian Cary Eclipse fluorescence spectrophotometer). Three independent experiments were performed and results correspond to a representative experiment with negative (PBS) and positive (melittin) controls.

SEM-FEG Imaging:

Scanning Electron Microscopy with Field Emission Gun (SEM-FEG) was used to obtain high-resolution images of the effect of guavanin 2 on the Gram-negative bacteria P. aeruginosa (ATCC 27853). Bacteria in mid-logarithmic phase were collected by centrifugation (100×g, 10 min, 4° C.), washed twice with PBS, and suspended in the same buffer at a density of 2×10⁷ CFU·mL⁻¹. 200 μL of the bacterial suspension were incubated 1 h at 37° C. with the peptide guavanin 2 at a final concentration corresponding to the MIC and 2-fold above the MIC. As a negative control, cells were incubated in buffer without peptide. Microbial cells were then fixed with 2.5% glutaraldehyde, homogenized by gently inverting the tubes and stored at 4° C. prior to SEM-FEG analysis. A Hitachi SU-70 Field Emission Gun Scanning Electron Microscope was used to record SEM-FEG images. The samples (gold plates where 20 μL of inoculum were deposited and dried under nitrogen) were fixed on an alumina SEM support with a carbon adhesive tape and were observed without metallization. In Lens Secondary electron detector (SE-Lower) was used to characterize the samples. The accelerating voltage was 1 kV and the working distance was around 15 mm. At least five to ten different locations were analyzed on each surface, leading to the observation of a minimum of 100 single cells.

Scarification Skin Infection Mouse Model: P. aeruginosa strain PAO1 was grown to an optical density at 600 nm (OD₆₀₀) of 1 in tryptic soy broth (TSB) medium. Subsequently cells were washed twice with sterile PBS, and resuspended to a final concentration of 5×10⁷ CFU/50 μL. To generate skin infection, female CD-1 mice (6 weeks old) were anesthetized with isoflurane and had their backs shaved. A superficial linear skin abrasion was made with a needle in order to damage the stratum corneum and upper-layer of the epidermis. Five minutes after wounding, an aliquot of 50 μL containing 5×10⁷ CFU of bacteria in PBS was inoculated over each defined area containing the scratch with a pipette tip. One day after the infection, peptides were administered to the infected area. Animals were euthanized and the area of scarified skin was excised two and four days post-infection, homogenized using a bead beater for 20 minutes (25 Hz), and serially diluted for CFU quantification. Two independent experiments were performed with 4 mice per group in each case. Statistical significance was assessed using a two-way ANOVA.

CD Spectroscopy:

Circular dichroism (CD) assays were carried out using JASCO J-815 spectropolarimeter equipped with a Peltier temperature controller (model PTC-423L/15). Measurements were recorded at 25° C. and performed in quartz cells of 1 mm path length between 195 and 260 nm at 0.2 nm intervals. Six repeat scans at a scan-rate of 50 nm·min⁻¹, 1 s response time and 1 nm bandwidth were averaged for each sample and for the baseline of the corresponding peptide-free sample. After subtracting the baseline from the sample spectra, CD data were processed with the Spectra Analysis software, which is part of Spectra Manager Platform. The relative helix content (H) according to the number of peptide bonds (n) was calculated from the ellipticity values at 222 nm as described by Chen et al. Biochemistry. 1974 Jul. 30; 13(16): 3350-9.

NMR Spectroscopy and Structure Calculations:

The NMR sample was prepared by dissolving guavanin 2 in a micellar solution containing 100 mM of deuterated dodecylphosphocholine (DPC-d₃₈), and 5% D₂O at 1 mM concentration. The pH was adjusted to 4.0. All spectra were acquired at 25° C. on a Bruker Avance III 500 spectrometer equipped with a 5 mm triple resonance broadband inverse (TBI) probehead. Proton chemical shifts were referenced to sodium 2,2-dimethyl-2-silapentane-5-sulfonate (DSS) and water suppression was achieved using the pre-saturation technique. ¹H-¹H TOCSY experiment was recorded with 128 transients of 4096 data points, 256 tl increments and a spinlock mixing time of 80 ms. The ¹H-¹H NOESY was recorded with 64 transients of 4096 data points, 256 tl increments, mixing time of 250 ms. Spectral width of 8012 Hz in both dimensions. ¹H-¹³C HSQC experiment was acquired with F1 and F2 spectral widths of 8012 and 25152 Hz, respectively were collected 256 tl increments with 96 transients of 4096 points for each free induction decay. The experiment was acquired in an edited mode. All NMR data were processed using NMRPIPE and analyzed with NMR View (Delaglio et al., J. Biomol. NMR. 1995 November; 6(3): 277-93; Johnson & Blevins, J. Biomol. NMR. 1994 September; 4(5): 603-614).

The structure calculations were performed with the XPLOR-NIH version 2.28 software by simulated annealing (SA) algorithm (Schwieters et al., J. Magn. Reson. 2003 January; 160(1): 65-73). NOE intensities were converted into semi-quantitative distance restrains using the calibration by Hyberts et al. Protein Sci. 1992 June; 1(6): 736-51. The angle restraints of phi and psi of the protein backbone dihedral angles were predicted based on analysis of ¹H_{α} and ¹³C_{α} chemical shifts using the program TALOS+ (Shen et al., J. Biomol. NMR. 2009 August; 44(4): 213-23). Several cycles of XPLOR were performed using standard protocols. After each cycle rejected restraints, side-chain assignments, NOEs and dihedral violations were analyzed. Two hundred structures were calculated, and among them, the 20 lowest energy structures were submitted to XPLOR-NIH water refinement protocol (Schwieters et al., J. Magn. Reson. 2003 January; 160(1): 65-73). The ensemble of the 10 lowest energy conformations was chosen to represent the solution structure ensemble of guavanin 2.

The restrictions used in structural calculations were analyzed by QUEEN program (Quantitative Evaluation of Experimental NMR Restraints). This program performs a quantitative assessment of the restrictions of the experimental NMR data. QUEEN checks and corrects possible assignments of errors by the analysis of the restrictions (Nabuurs et al., J. Am. Chem. Soc. 2003 Oct. 1; 125(39): 12026-12034). The stereochemical quality of the lowest energy structures was analyzed by PROCHECK and ProSA (Wiederstein & Sippl, Nucleic Acids Res. 2007 May 21; 35: W407-10; Laskowski et al., J. Appl. Cryst. 1993; 26: 283-291). PROCHECK was used in order to check stereochemical quality of protein structure through the Ramachandran plot, where good quality models are expected to have more than 90% of amino acid residues in most favored and additional allowed regions. ProSA indicates the fold quality by means of the Z-score. The display, analysis, and manipulation of the three-dimensional structures were performed with the program MOLMOL (Koradi et al., J. Mol. Graph. 1996 February; 14(1): 51-5, 29-32) and PyMOL (The PyMOL Molecular Graphics System, Version 1.8 Schridinger, LLC).

Solvation Potential Energy Calculation:

The solvation potential energy was measured for the ten lower energy NMR structures. Each structure was separated into a single pdb file. The conversion of pdb files into pqr files was perfomed by the utility PDB2PQR using the AMBER force field (Dolinsky et al., Nucleic Acids Res. 2004 Jul. 1; 32: W665-7). The grid dimensions for Adaptive Poisson-Boltzmann Solver (APBS) calculation were also determined by PDB2PQR. Solvation potential energy was calculated by APBS (Baker et al., Proc. Natl. Acad. Sci. U.S.A. 2001 Aug. 21; 98(18): 10037-41). Surface visualization was performed using the APBS plugin for PyMOL.

### Example 8. Discussion

AMPs represent promising alternatives to conventional antibiotics to combat the global health problem of antibiotic resistance. Their development has been slowed, however, by a lack of methods that would enable their cost-effective and rational design. Here, a computational platform is described that can be used to generate in silico peptides with antimicrobial properties by harnessing principles from biological evolution. Since peptides are built computationally and ranked according to their fitness function scores, only those “artificially evolved” peptides ranked highest are subsequently synthesized chemically, thus reducing experimental costs. In addition, the platform generates unique sequences that do not exist in nature. In particular, the focus was the re-design of the plant peptide Pg-AMP1. The first plant AMPs were identified in the 1970s; since that time, a number of classes of AMPs have been identified (Candido et al. (ed. Méndez-Vilas, A.) 951-960 (Formatex, 2011)). These plant AMPs are composed of tens of amino acids residues and have an uncommon composition and structures stabilized by disulfide bridges. The complexity of their chemical structures is perhaps the main disadvantage of plant-based AMPs and likely is one reason that none have reached the market (Candido et al. (ed. Méndez-Vilas, A.) 951-960 (Formatex, 2011)). Promising design methods have recently been applied to engineer AMPs and help overcome such limitations while simultaneously increasing AMP potency and reducing cytotoxicity towards human cells (Porto et al. (ed. Faraggi, E.) 377-396 (InTech, 2012). doi: 10.5772/2335). Unfortunately, many of these methods are based on incremental modifications of an AMP template, which is costly, and when new peptides are designed from scratch, they often share similarity with AMP sequences found in databases. Consequently, only a very limited set of amino acids is harnessed to design the “new” AMPs.

In the present study, a computer-aided design platform is described for exploring the sequence space of AMPs and generating innovative “artificial” AMPs. A custom GA was leveraged to optimize the guava plant peptide Pg-AMP1 and generated the synthetic guavanin peptides, several of which displayed potent activity against the Gram-negative pathogen P. aeruginosa. The application of GAs is not a novelty in the field of AMP design (Maccari et al., PLoS Comput. Biol. 2013 Sep. 5; 9(9): e1003212; Patel et al., J. Comput. Aided. Mol. Des. 1998 November; 12(6): 543-56; Fjell et al., Chem. Biol. Drug Des. 2010 Oct. 13; 77(1): 48-56). However, the custom GA presents two main important modifications for designing truly innovative peptides: (i) the application of an equation, instead of a machine learning classifier, and (ii) the interruption of the algorithm before it reaches plateau, which enables exploration of unconventional sequence space.

The fitness function was implemented as an equation that relates hydrophobic moment and α-helical propensity; thus, it guides the algorithm to select amphipathic and α-helical peptides but not necessarily sequences that correspond to traditional AMPs, which explains the generation of several peptides with modest antimicrobial activity (TABLEs 3 and 4). Owing to the improvement in the hydrophobic moment, two kinds of amino acids would be preferentially selected during the iteration steps: both positively charged (mainly Arg residues) and hydrophobic residues (Leu and Ile residues). Therefore, the application of the fitness function should favor a peptide with a segregation of positively charged and hydrophobic residues that adopts an α-helical structure in hydrophobic environments, characteristic of many conventional AMPs (Brogden, Nat. Rev. Microbiol. 2005 March; 3(3): 238-50; Fjell et al., Nat. Rev. Drug Discov. 2011 Dec. 16; 11(1): 37-51; Porto et al., (ed. Faraggi, E.) 377-396 (InTech, 2012). doi:10.5772/2335).

After hundreds of algorithm iterations, an optimal solution to this type of mathematical modeling would result in peptides composed primarily of Ala, Arg, Ile and Lys residues [as observed by Patel et al. (J. Comput. Aided. Mol. Des. 1998 November; 12(6): 543-56) and Maccari et al. (PLoS Comput. Biol. 2013 Sep. 5; 9(9): e1003212). However, in order to obtain peptides with uncommon amino acid composition that do not exist in nature, the algorithm was set to promote slow optimization (200 of 250 sequences were promoted to the next iteration and a low mutation rate of 0.05% was allowed), and the iterations were stopped before the fitness function plateaued (FIG. 1C). Therefore, a suboptimal solution was reached for the mathematical model in order to generate peptide sequences that exhibited unique amino acid compositions compared to sequences found in the APD (FIG. 1D).

The computationally designed guavanins were found to be rich in arginine residues (and some of them are also tyrosine-rich), whereas the parent peptide, Pg-AMP1, is classified as a glycine-rich peptide; four Pg-AMP1 fragments were used in the founder population (FIGS. 1A and B) and three of them were rich in tyrosine residues (FIG. 5). During the algorithm iterations, Gly residues tended to disappear, as they do not favor α-helix formation (FIG. 5). Conversely, Arg residues were rapidly fixed in the derived populations, as this residue serves as the cationic counterpart of the peptide and has a good α-helical propensity (FIG. 5). As the algorithm promoted slow optimization, Tyr residues were retained as the hydrophobic counterpart of the peptide; however, there would be a tendency to replace them by Leu or Ile residues with more iteration steps if the fitness function had been allowed to reach a plateau (FIG. 5). Ultimately, owing to the slow optimization process, the most active peptide, guavanin 2, possessed a residual Gly residue and only four accumulated mutations (FIG. 5).

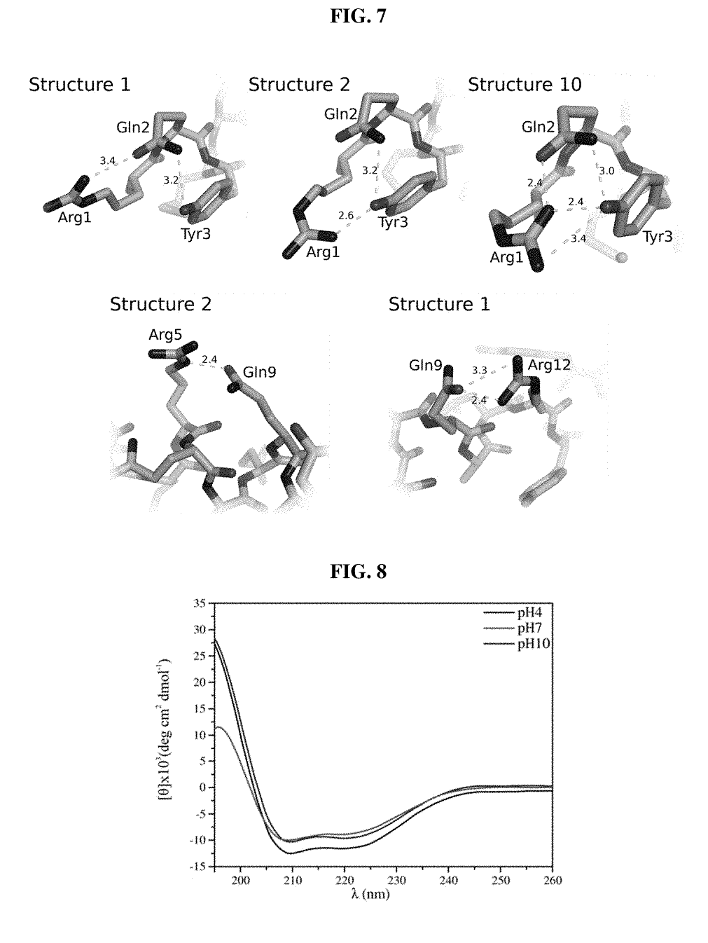

This approach resulted in eight novel AMPs, out of the fifteen guavanins (53%) characterized, following the criteria of classification of peptides as antimicrobial (TABLE 3). These eight AMPs had lower MIC values against P. aeruginosa than the four Pg-AMP1 fragments used as starting peptide sequences (TABLE 3). Four of the “artificial” peptides generated (guavanins 2, 12, 13 and 14) also displayed lower MICs vs. P. aeruginosa (TABLE 3) compared to the original natural peptide Pg-AMP1 (MIC of 100 μg mL⁻¹). In addition, all the modeled guavanins were predicted to form an α-helical secondary structure (FIG. 6 and FIG. 7).

Structural studies of lead peptide guavanin 2, performed using CD and NMR spectroscopy, demonstrated that the approach had successfully generated an α-helical peptide. The CD studies indicated that guavanin 2 was unstructured in aqueous solution, but formed a well-defined α-helical structure in the presence of micelles or structure-inducing solvents (FIG. 3A). The NMR analysis revealed that guavanin 2 formed an ca-helical structure between residues Gln²-Arg¹⁶ in the presence of 100 mM DPC-d₃₈ micelles, further supporting the CD structural data and suggesting that guavanin 2 adopts a predominantly α-helical conformation in the presence of a biological membrane.

Further characterization of the biological properties of guavanin 2 revealed that this peptide acted preferentially against Gram-negative bacteria (TABLE 3), and had a selectivity index of 23.93. As this index is analogous to the therapeutic index, guavanin 2 may be considered a safe peptide based on the in vitro results: according to the U.S. Food and Drug Administration, a therapeutic index is considered narrow when it is below two, while for a safer drug, the higher the index, the better the drug (Muller & Milton, Nat. Rev. Drug Discov. 2012 Aug. 31; 11(10): 751-61). The selectivity index value could also be considered as an improvement, since recombinant Pg-AMP1 and the charged fragment display indices of 4.88 and 0.5, respectively (Pelegrini et al., Peptides. 2008 Mar. 22; 29(8): 1271-9). Therefore, the pharmacological properties of guavanin 2 were superior to that of Pg-AMP1, as guavanin 2 was almost five times safer as well as three times smaller than Pg-AMP1, while the charged fragment was considered toxic. Because Pg-AMP1 is hemolytic (Pelegrini et al., Peptides. 2008 Mar. 22; 29(8): 1271-9), as well as its 2^{nd} fragment (TABLE 3), their use is limited to non-intravenous use. Therefore, their anti-infective potential was assessed using an abscess infection model. These experiments revealed that at a low dose of 6.25 μg mL⁻¹, guavanin 2 was superior to its predecessors Pg-AMP1 and Pg-AMP1 fragment 2, consistent with the in vitro MIC results. Previously, the effects of Cycloviolacin O2 and Kalata B2 were demonstrated against S. aureus using a similar in vivo model (Fensterseifer et al., Peptides. 2014 Nov. 8; 63: 38-42). Since guavanin 2 is a linear peptide, it has the advantage of ease of synthesis compared with cyclotides that require post-translational modifications to achieve their active form (Pinto et al., Complementary Altern. Med. 2011 Dec. 15; 17, 40-53).

Since guavanin 2 is a new AMP, its mechanism of action was investigated. As described herein, this peptide kills E. coli cells but does so slowly, similarly to temporin-SHd (Abbassi et al., Biochimie. 2012 Oct. 29; 95(2): 388-99). In addition, SEM-FEG imaging indicated that guavanin 2 induces bacterial membrane damage (FIG. 2B). It is important to highlight that the membranolytic activity of guavanin 2 is different from that of melittin and the recently designed peptide [Is, R⁸] mastoparan (Irazazabal et al., Biochim. Biophys. Acta. 2016 Jul. 14; 1858(11): 2699-2708). For guavanin 2, the killing was 8-fold slower than for [Is, R⁸] mastoparan, and guavanin 2 also slowly permeated the cytoplasmic membrane by inducing membrane hyperpolarization, in contrast to melittin (FIG. 2A). In fact, the hyperpolarization indicates that guavanin 2 could act as a selective ionophore, similar to the antimicrobial compounds valinomycin and citral (Schiefer et al., Curr. Microbiol. 1979 March; 3: 85-88; Shi et al., PLoS One. 2016 Jul. 14; 11(7): e0159006), which are selective for potassium ions. Altogether, these results suggest that the potent effect of guavanin 2 observed against P. aeruginosa (TABLEs 3 and 4) is due to pore formation within the cytoplasmic membrane.

Despite the previous demonstration of peptide magainin G inducing hyperpolarization on tumor cells and of PAF peptide and Rs-AFP2 on fungal cells (Cruciani et al., Proc. Natl. Acad. Sci. U.S.A. 1991 May 1; 88(9): 3792-6; Marx et al., Cell. Mol. Life Sci. 2008 February; 65(3): 445-454; Thevissen et al., Appl. Environ. Microbiol. 1999 December; 65(12): 5451-8), this is the first demonstration of bacterial membrane hyperpolarization driven by a polypeptide. Such an effect could reflect the amino acid composition: guavanin 2 contains 30% arginine residues as well as uncommon amino acids for AMPs such as tyrosine and glutamine residues, having 3 of each. These results indicate that the inclusion of non-proteinogenic amino acids (e.g. norleucine, ornithine) is not essential to obtaining innovative peptides (Maccari et al., PLoS Comput. Biol. 2013 Sep. 5; 9(9): e1003212; Giangaspero et al., Eur. J. Biochem. 2001 November; 268(21): 5589-600). In fact, it is difficult to escape from the utilization of Arg or Lys residues, even though some AMPs include His residues (Park et al., Plant Mol. Biol. 2000 September; 44(2): 187-97), as these are the only natural residues that possess positively charged side chains. In addition, it was demonstrated that it is possible to use hydrophobic residues other than Trp and Phe (the most abundant in naturally occurring sequences). In the case of guavanin 2, Tyr is the hydrophobic counterpart of the peptide.

In the present study, a novel AMP, guavanin 2 has been evolved in silico and optimized. It was demonstrated that guavanin 2 is a better candidate for drug development than the naturally occurring peptide, Pg-AMP1. It was also demonstrated that naturally occurring peptides, such as those derived from plants, may serve as excellent templates for identifying novel AMP sequences with therapeutic potential. Guavanin 2 has an unusual mechanism of action, as it causes membrane hyperpolarization, whereas other peptides depolarize it. Manipulation of natural AMP sequences using the computational platform described here may be used to explore peptide sequence space and uncover innovative combinations of amino acids that may lead to the development of designed AMPs with distinct mechanisms of action and biological potency.

### REFERENCES

1. Peleg A. Y. & Hooper D. C., Hospital-acquired infections due to gram-negative bacteria. N. Engl. J. Med. 2010 May 13; 362(19): 1804-13.
2. Gaynes R. & Edwards J. R., Overview of nosocomial infections caused by gram-negative bacilli. Clin. Infect. Dis. 2005 Sep. 15; 41(6): 848-54.
3. Fleischmann C., Scherag A., Adhikari N. K., Hartog C. S., Tsaganos T., Schlittmann P., Angus D. C., & Reinhart K., Assessment of Global Incidence and Mortality of Hospital-treated Sepsis. Current Estimates and Limitations. Am. J. Respir. Crit. Care Med. 2016 Feb. 1; 193(3): 259-72.
4. Coates A. R. M., Halls G., & Hu Y. Novel classes of antibiotics or more of the same? Br. J. Pharmacol. 2011 May; 163(1): 184-94.
5. Brogden K. A., Antimicrobial peptides: pore formers or metabolic inhibitors in bacteria? Nat. Rev. Microbiol. 2005 March; 3(3): 238-50.
6. Fjell C. D., Hiss J. A., Hancock R. E., & Schneider G. Designing antimicrobial peptides: form follows function. Nat. Rev. Drug Discov. 2011 Dec. 16; 11(1): 37-51.
7. Candido E. S. et al. in Science against Microbial Pathogens: Communicating Current Research and Technological Advances (ed. Méndez-Vilas, A.) 951-960 (Formatex, 2011). at formatex.info/microbiology3/book/951-960.pdf
8. Harris P. W. R., Yang S. H., Molina A., Lopez G., Middleditch M., & Brimble M. A., Plant antimicrobial peptides snakin-1 and snakin-2: chemical synthesis and insights into the disulfide connectivity. Chemistry. 2014 Mar. 8; 20(17): 5102-10.
9. Cheneval O., Schroeder C. I., Durek T., Walsh P., Huang Y. H., Liras S., Price D. A., & Craik D. J., Fmoc-based synthesis of disulfide-rich cyclic peptides. J. Org. Chem. 2014 Jun. 11; 79(12): 5538-44.
10. Porto W. F., Silva O. N., & Franco, O. L. in Protein Structure (ed. Faraggi, E.) 377-396 (InTech, 2012). doi:10.5772/2335.
11. Diller D. J., Swanson J., Bayden A. S., Jarosinski M., & Audie J., Rational, computer-enabled peptide drug design: principles, methods, applications and future directions. Future Med. Chem. 2015 Oct. 29; 7(16): 2173-93.
12. Thennarasu S. & Nagaraj R., Specific antimicrobial and hemolytic activities of 18-residue peptides derived from the amino terminal region of the toxin pardaxin. Protein Eng. 1996 December; 9(12): 1219-24.
13. Cardoso M. H., Ribeiro S. M., Nolasco D. O., de la Fuente-Nunez C., Felicio M. R., Gonclaves S., Matos C. O., Liao L. M., Santos N. C., Hancock R. E., Franco O. L., & Migliolo L., A polyalanine peptide derived from polar fish with anti-infectious activities. Sci. Rep. 2016 Feb. 26; 6: 21385.
14. Loose C., Jensen K., Rigoutsos I., & Stephanopoulos G., A linguistic model for the rational design of antimicrobial peptides. Nature. 2006 Oct. 19; 443(7): 867-9.
15. Maccari G., Di Luca M., Nifosi R., Cardarelli F., Signore G., Boccardi C., & Bifone A., Antimicrobial peptides design by evolutionary multiobjective optimization. PLoS Comput. Biol. 2013 Sep. 5; 9(9): e1003212.
16. Porto W. F., Pires A. S., & Franco, O. L. Antimicrobial activity predictors benchmarking analysis using shuffled and designed synthetic peptides. J. Theor. Biol. 2017 May 20; 426: 96-103.
17. Patel S., Stott I. P., Bhakoo M., & Elliott P., Patenting computer-designed peptides. J. Comput. Aided. Mol. Des. 1998 November; 12(6): 543-56.
18. Fjell C. D., Jenssen H., Cheung W. A., Hancock R. E., & Cherkasov A., Optimization of antibacterial peptides by genetic algorithms and cheminformatics. Chem. Biol. Drug Des. 2010 Oct. 13; 77(1): 48-56.
19. Giangaspero A., Sandri L., & Tossi A., Amphipathic alpha helical antimicrobial peptides. Eur. J. Biochem. 2001 November; 268(21): 5589-600.
20. Pelegrini P. B., Murad A. M., Silva L. P., Dos Santos R. C., Costa F. T., Tagliari P. D., Bloch C. Jr., Noronha E. F., Miller R. N., & Franco O. L., Identification of a novel storage glycine-rich peptide from guava (Psidium guajava) seeds with activity against Gram-negative bacteria. Peptides. 2008 Mar. 22; 29(8): 1271-9.
21. Park C. J., Park C. B., Hong S. S., Lee H. S., Lee S. Y., & Kim S. C., Characterization and cDNA cloning of two glycine- and histidine-rich antimicrobial peptides from the roots of shepherd's purse, Capsella bursa-pastoris. Plant Mol. Biol. 2000 September; 44(2): 187-97.
22. Cao H., Ke T., Liu R., Yu J., Dong C., Cheng M., Huang J., & Liu S., Identification of a Novel Proline-Rich Antimicrobial Peptide from Brassica napus. PLoS One. 2015 Sep. 18; 10(9): e0137414.
23. Winkler D. F., Hilpert K., Brandt O., & Hancock R. E., Synthesis of peptide arrays using SPOT-technology and the CelluSpots-method. Methods Mol. Biol. 2009; 570: 157-74.
24. Tavares L. S., Rettore J. V., Freitas R. M., Porto W. F., Dugue A. P., Singuani J. L., Silva O. N., Detoni M. L., Vasconcelos E. G., Dias S. C., Franco O. L., & Santos M. O., Antimicrobial activity of recombinant Pg-AMP1, a glycine-rich peptide from guava seeds. Peptides. 2012 Jul. 27; 37(2): 294-300.
25. Irazazabal L. N., Porto W. F., Ribeiro S. M., Casale S., Humblot V., Ladram A., & Franco O. L., Selective amino acid substitution reduces cytotoxicity of the antimicrobial peptide mastoparan. Biochim. Biophys. Acta. 2016 Jul. 14; 1858(11): 2699-2708.
26. Rex S., Pore formation induced by the peptide melittin in different lipid vesicle membranes. Biophys. Chem. 1996 Jan. 16; 58(1-2): 75-85.
27. Wang G., Determination of solution structure and lipid micelle location of an engineered membrane peptide by using one NMR experiment and one sample. Biochim. Biophys. Acta. 2007 December; 1768(12): 3271-81.
28. Usachev K. S., Efimov S. V, Kolosova O. A., Filippov A. V., & Klochkov V. V., High-resolution NMR structure of the antimicrobial peptide protegrin-2 in the presence of DPC micelles. J. Biomol. NMR. 2014 Nov. 28; 61(3-4): 227-34.
29. Muller P. Y. & Milton M. N., The determination and interpretation of the therapeutic index in drug development. Nat. Rev. Drug Discov. 2012 Aug. 31; 11(10): 751-61.
30. Fensterseifer I. C., Silva O. N., Malik U., Ravipati A. S., Novaes N. R., Miranda P. R., Rodrigues E. A., Moreno S. E., Craik D. J., & Franco O. L., Effects of cyclotides against cutaneous infections caused by Staphylococcus aureus. Peptides. 2014 Nov. 8; 63: 38-42.
31. Pinto M. F. S., Almeida R. G., Porto W. F., Fensterseifer I. C. M., Lima L. A., Dias S. C>, & Franco O. L., Cyclotides: From Gene Structure to Promiscuous Multifunctionality. J. Evid. Based. Complementary Altern. Med. 2011 Dec. 15; 17, 40-53.
32. Abbassi F., Raja Z., Oury b., Gazanion E., Piesse C., Sereno D., Nicolas P., Foulon T., & Ladram A., Antibacterial and leishmanicidal activities of temporin-SHd, a 17-residue long membrane-damaging peptide. Biochimie. 2012 Oct. 29; 95(2): 388-99.
33. Schiefer H. G., Schummer U., & Gerhardt U., Effect of cell membrane-active antimicrobial agents on membrane potential and viability of mycoplasmas. Curr. Microbiol. 1979 March; 3: 85-88.
34. Shi C., Song K., Zhang X., Sun Y., Sui Y., Chen Y., Jia Z., Sun H., Sun Z., & Xia X., Antimicrobial Activity and Possible Mechanism of Action of Citral against Cronobacter sakazakii. PLoS One. 2016 Jul. 14; 11(7): e0159006.
35. Cruciani R. A., Barker J. L., Zasloff M., Chen H. C., & Colamonici O., Antibiotic magainins exert cytolytic activity against transformed cell lines through channel formation. Proc. Natl. Acad. Sci. U.S.A. 1991 May 1; 88(9): 3792-6.
36. Marx F., Binder U., Leiter É., & Pócsi I., The Penicillium chrysogenum antifungal protein PAF, a promising tool for the development of new antifungal therapies and fungal cell biology studies. Cell. Mol. Life Sci. 2008 February; 65(3): 445-454.
37. Thevissen K., Terras F. R., & Broekaert W. F., Permeabilization of fungal membranes by plant defensins inhibits fungal growth. Appl. Environ. Microbiol. 1999 December; 65(12): 5451-8.
38. Pace C. N. & Scholtz J. M., A Helix Propensity Scale Based on Experimental Studies of Peptides and Proteins. Biophys. J. 1998 July; 75(1): 422-427.
39. Porto W. F., Nolasco D. O., & Franco O. L., Native and recombinant Pg-AMP1 show different antibacterial activity spectrum but similar folding behavior. Peptides. 2014 Feb. 26; 55: 92-7.
40. Eisenberg D., Weiss R. M., Terwilliger T. C., & Wilcox W., Hydrophobic moments and protein structure. Faraday Symp. Chem. Soc. 1982; 17, 109.
41. Wang G., Li X., & Wang Z., APD2: the updated antimicrobial peptide database and its application in peptide design. Nucleic Acids Res. 2008 Oct. 28; 37: D933-7.
42. Hammami R., Ben Hamida J., Vergoten G., & Fliss I., PhytAMP: a database dedicated to antimicrobial plant peptides. Nucleic Acids Res. 2008 Oct. 4; 37: D963-8.
43. Xu D. & Zhang Y., Ab initio protein structure assembly using continuous structure fragments and optimized knowledge-based force field. Proteins. 2012 Apr. 13; 80(7): 1715-35.
44. Wiederstein M. & Sippl M. J., ProSA-web: interactive web service for the recognition of errors in three-dimensional structures of proteins. Nucleic Acids Res. 2007 May 21; 35: W407-10.
45. Laskowski R., Macarthur M., Moss D., & Thornton J., PROCHECK: a program to check the stereochemical quality of protein structures. J. Appl. Cryst. 1993; 26: 283-291.
46. Webb B. & Sali A., Comparative Protein Structure Modeling Using MODELLER. Curr. Protoc. Bioinformatics. 2014 Sep. 8; 47: 5.6.1-5.6.32.
47. Hilpert K. & Hancock R. E., Use of luminescent bacteria for rapid screening and characterization of short cationic antimicrobial peptides synthesized on cellulose using peptide array technology. Nat. Protoc. 2007; 2(7): 1652-60.
48. Abbassi F., Oury B., Blasco T., Sereno D., Bolbach G., Nicolas P., Hani K., Amiche M., & Ladram A., Isolation, characterization and molecular cloning of new temporins from the skin of the North African ranid Pelophylax saharica. Peptides. 2008 September; 29(9): 1526-33.
49. Riss T. et al. in Assay Guidance Manual (eds. Sittampalam, G. et al.) (Bethesda (Md.), 2004). at europepmc.org/books/NBK144065
50. Chen Y., Mant C. T., Farmer S. W., Hancock R. E., Vasil M. L., & Hodges R. S., et al. Rational design of alpha-helical antimicrobial peptides with enhanced activities and specificity/therapeutic index. J. Biol. Chem. 2005 Apr. 1; 280(13): 12316-29.
51. Sims P. J., Waggoner A. S., Wang C. H., & Hoffman J. F., Studies on the mechanism by which cyanine dyes measure membrane potential in red blood cells and phosphatidylcholine vesicles. Biochemistry. 1974 Jul. 30; 13(16): 3315-30.
52. André S., Washington S. K., Darby E., Bega M. M., Filip A. D., Ash N. S., Muzikar K. A., Piesse C., Foulon T., O'Leary D. J., & Ladram A., Structure-Activity Relationship-based Optimization of Small Temporin-SHf Analogs with Potent Antibacterial Activity. ACS Chem. Biol. 2015 Jul. 30; 10(10): 2257-66.
53. Chen Y. H., Yang J. T., & Chau K. H., Determination of the helix and beta form of proteins in aqueous solution by circular dichroism. Biochemistry. 1974 Jul. 30; 13(16): 3350-9.
54. Delaglio F., Grzesiek S., Vuister G. W., Zhu G., Pfeifer J., & Bax A., NMRPipe: a multidimensional spectral processing system based on UNIX pipes. J. Biomol. NMR. 1995 November; 6(3): 277-93.
55. Johnson B. A. & Blevins R. A., NMR View: A computer program for the visualization and analysis of NMR data. J. Biomol. NMR. 1994 September; 4(5): 603-614.
56. Schwieters C. D., Kuszewski J. J., Tjandra N., & Clore G. M., The Xplor-NIH NMR molecular structure determination package. J. Magn. Reson. 2003 January; 160(1): 65-73.
57. Hyberts S. G., Goldberg M. S., Havel T. F., & Wagner G., The solution structure of eglin c based on measurements of many NOEs and coupling constants and its comparison with X-ray structures. Protein Sci. 1992 June; 1(6): 736-51.
58. Shen Y., Delaglio F., Cornilescu G., & Bax A., TALOS+: a hybrid method for predicting protein backbone torsion angles from NMR chemical shifts. J. Biomol. NMR. 2009 August; 44(4): 213-23.
59. Nabuurs S. B., Spronk C. A., Krieger E., Maassen H., Vriend G., & Vuister G. W., Quantitative Evaluation of Experimental NMR Restraints. J. Am. Chem. Soc. 2003 Oct. 1; 125(39): 12026-12034.
60. Koradi R., Billeter M., & Wiithrich K., MOLMOL: a program for display and analysis of macromolecular structures. J. Mol. Graph. 1996 February; 14(1): 51-5, 29-32.
61. Dolinsky T. J., Nielsen J. E., McCammon J. A., & Baker N. A., PDB2PQR: an automated pipeline for the setup of Poisson-Boltzmann electrostatics calculations. Nucleic Acids Res. 2004 Jul. 1; 32: W665-7.
62. Baker N. A., Sept D., Joseph S., Holst M. J., & McCammon J. A., Electrostatics of nanosystems: application to microtubules and the ribosome. Proc. Natl. Acad. Sci. U.S.A. 2001 Aug. 21; 98(18): 10037-41 (2001).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

1. A method of designing peptides having at least one property of interest, said method comprising:
a. selecting a population of parent peptides;
b. calculating a fitness function value for each peptide in the population of peptides of (a), wherein the fitness function value is indicative of the presence of at least one property of interest;
c. selecting a fraction of the peptides from the population of peptides, wherein the fitness function values of the selected fraction of peptides are higher than the fitness function values of the non-selected fraction of peptides;
d. subjecting the fraction of peptides in (c) to fitness-guided mutation comprising at least a single point cross over and at least a 0.05% probability of mutation, thereby generating a population of mutated peptides;
e. calculating a fitness function value for each peptide in the population of mutated peptides of (d), wherein the fitness function value is indicative of the presence of the at least one property of interest in (b); and
f. iteratively repeating steps (c)-(e), wherein the number of iterations does not result in the plateauing of the average fitness function values of the population of selected peptides of (e).

2. The method of claim 1, wherein the peptides in the population of parent peptides in (a) consist of the same amino acid sequence.

3. The method of claim 1, wherein the peptides in the population of parent peptides in (a) comprise two or more amino acid sequences.

4. The method of claim 1, wherein each peptide in the population of parent peptides in (a) has essentially the same fitness function value.

5. The method of claim 4, wherein the fitness function is represented by the equation:
$$Fitness = \frac{\sqrt[2]{{[ {\sum }_{i = 1}^{I} {H}_{i} \times cos ( δ i ) ]}^{2} + {[ {\sum }_{i = 1}^{I} {H}_{i} \times sin ( δ i ) ]}^{2}}}{{\sum }_{i = 1}^{I} {e}^{{Hx}_{i}}}$$
where δ represents the angle between the amino acid side chains; i represents the residue number in the position i from the sequence; Hi represents the ith amino acid's hydrophobicity on a hydrophobicity scale; Hxi represents the ith amino acid's helix propensity in Pace-Schols scale; and I represents the total number of residues present in the sequence.

6. The method of claim 3, wherein, prior to step (b), the peptides in the population of parent peptides are subject to random crossing over between the peptides in the population.

7. The method of claim 1, wherein the amino acid sequence of at least one of the peptides in the population of peptides comprises the amino acid sequence of an antimicrobial peptide (AMP) or an AMP fragment.

8.-9. (canceled)

10. The method of claim 1, wherein the fraction of peptides selected from the population in (c) comprises at least 250 unique amino acid sequences.

11. The method of claim 1, wherein the non-selected fraction of peptides in (c) comprise amino acid sequences corresponding to the 50 worst fitness values calculated in (b) or (e).

12. The method of claim 1, wherein at least one of the at least one property of interest is selected from the group consisting of α-helical propensity, higher net charge, hydrophobicity, and hydrophobic moment.

13. The method of claim 1, wherein the fitness function in (b) or (e) is represented by the equation:
$$Fitness = \frac{\sqrt[2]{{[ {\sum }_{i = 1}^{I} {H}_{i} \times cos ( δ i ) ]}^{2} + {[ {\sum }_{i = 1}^{I} {H}_{i} \times sin ( δ i ) ]}^{2}}}{{\sum }_{i = 1}^{I} {e}^{{Hx}_{i}}}$$
where δ represents the angle between the amino acid side chains; i represents the residue number in the position i from the sequence; Hi represents the ith amino acid's hydrophobicity on a hydrophobicity scale; Hxi represents the ith amino acid's helix propensity in Pace-Schols scale; and I represents the total number of residues present in the sequence.

14. An antimicrobial peptide (AMP) designed according to the method of claim 1.

15. The AMP of claim 14, wherein the AMP has a minimal inhibitory concentration (MIC) that is lower than or equal to the peptide from which it was derived.

16. An antimicrobial peptide (AMP) comprising the amino acid sequence of any one of SEQ ID NOs: 1-100.

17. The AMP of claim 16, wherein the antimicrobial peptide comprises the amino acid sequence RQYMRQIEQALRYGYRISRR (SEQ ID NO: 2) from N-terminal to C-terminal.

18. A composition comprising the antimicrobial peptide of claim 14, optionally further comprising a pharmaceutically acceptable carrier and/or excipient.

19. A method of treating a patient having a bacterial infection comprising administering an AMP of claim 14 to the patient.

20. The method of claim 19, wherein the bacterial infection is a gram-negative bacterial infection, optionally wherein the gram-negative bacteria is selected from the group consisting of Escherichia coli, Pseudomonas aeruginosa, Klebsiella pneumonia, Acinetobacter baumanii, and Neisseria gonorrhoeae.

21. (canceled)

22. The method of claim 7, wherein the AMP or AMP fragment is a plant AMP or a plant AMP fragment, optionally Pg-AMP1 or a Pg-AMP1 fragment.

23. The method of claim 22, wherein the AMP or AMP fragment is a Pg-AMP1 fragment, wherein the Pg-AMP1 fragment is Pg-AMP1 fragment 2.
